(19) United States
(12) Patent Application Publication
(10) Pub. No.: US 2022/0315940 A1
(43) Pub. Date: Oct. 6, 2022

(54) GERMACRENE A SYNTHASE MUTANTS

(71) Applicants: Keygene N.V., Wageningen (NL); Stichting Wageningen Research, Wageningen (NL)

(72) Inventors: Hendrik Jan BOSCH, Wageningen (NL); Michiel Theodoor Jan DE BOTH, Wageningen (NL); Katarina CANKAR, Wageningen (NL); Paul BUNDOCK, Wageningen (NL); Robert Edouard SEVENIER, Wageningen (NL); Martinus Julius BEEKWILDER, Wageningen (NL)

(21) Appl. No.: 17/843,627

(22) Filed: Jun. 17, 2022

Related U.S. Application Data
(63) Continuation of application No. PCT/EP2020/086755, filed on Dec. 17, 2020.

(30) Foreign Application Priority Data
Dec. 19, 2019 (EP) 19217865.5
Jun. 15, 2020 (EP) 20180022.4

(51) Int. Cl. C12N 15/82; C12N 15/11; C12N 9/22; C12N 9/88

(52) U.S. Cl. CPC C12N 15/8243; C12N 15/8213; C12N 15/11; C12N 9/22; C12N 9/88; C12N 2310/20; C12N 2800/80

(57) ABSTRACT

The invention is in the field of agriculture, in particular in the field of crop improvement for processing, more particularly in the field of sesquiterpene lactone (STL), squalene and phenolic compound biosynthesis by plants. A method for producing a plant having reduced STL levels, increased squalene levels and increased phenolic compound levels is disclosed, as well as a plant produced by such method.

### CROSS-REFERENCE TO RELATED APPLICATIONS

The present application is a Continuation of International Patent Application No. PCT/EP2020/086755, filed Dec. 17, 2020, which claims priority to: Europe Patent Application No. 19217865.5 filed Dec. 19, 2019, and Europe Patent Application No. 20180022.4 filed Jun. 15, 2020, the entire contents of both of which are hereby incorporated by reference.

### SEQUENCE LISTING

The instant application contains a Sequence Listing which has been submitted electronically in ASCII format and is hereby incorporated by reference in its entirety. Said ASCII copy, created on Jun. 16, 2022, is named 085342-4400_SequenceListing.txt and is 82,296 bytes in size.

### FIELD OF THE INVENTION

The invention is in the field of agriculture, in particular in the field of crop improvement for processing, more particularly in the field of sesquiterpene lactones biosynthesis in plants.

### BACKGROUND OF THE INVENTION

Chicory (Cichorium intybus L.) is a perennial plant from the Asteraceae family which forms a strong taproot allowing the plant to persist during periods of drought and temperature stress. C. intybus is grown for many different applications, and is divided into several different varieties according to their use. Various cultivars cultivated for their leaves are all grouped into C. intybus var. foliosum. From this group Belgian endive is cultivated as a vegetable predominantly in the regions of northern France, Belgium and The Netherlands as a etiolated compact leaf structure, as white “witlof”. The related species C. endivia is consumed as a green leafy vegetable (endive). Raddichio varieties with the typical red crops also belong to the C. intybus var. foliosum. These different forms of vegetables are appreciated for their bitter taste. The taproot of another variety, C. intybus var. sativa, is grown for an industrial application, the isolation of inulin. Inulin is a fructose polymer which is used as both a soluble food fibre and also as a low calorie sweetener which is finding increasing applications in low sugar products. One reason for this robust growth is the presence of the bitter compounds in the leaves and roots, which belong to the class of sesquiterpene lactones (STLs).

STLs are a class of plant secondary metabolites that predominantly occur in the plant species of the Asteraceae family. They have been shown to have a variety of bioactivities, ranging from allelopathic activity and protective activity against herbivorous insects in roots and flowers (Molinaro et al. J. Environ. Sci. Health B 2016, 51, 847-852; Huber et al. PLoS Biol. 2016, 14, e1002332; Prasifka et al. J. Agric. Food Chem 2015, 63, 4042-4049). In chicory, STLs provide bitterness to the vegetables and the roots, which have also been used as a coffee substitute. The STLs in the root are also co-isolated with inulin and then have to be subsequently removed with additional purification steps, increasing the cost of inulin isolation. The major STLs of chicory belong to the class of guaianolide sesquiterpene lactones and are thought to be derived from a single common sesquiterpene, germacrene A. In additional biosynthesis steps, germacrene A is further modified (e.g. through oxidations, lactone ring closures and conjugations to oxalate, hydroxyphenylacetate and/or glycosyl moieties) to yield a variety of guaianolide sesquiterpene structure to diversify their biological properties. In chicory the most predominant STLs are lactucin, lactucopicrin and 8-deoxylactucin, including their oxalates (Sessa et al. J. Biol. Chem. 2000, 275, 26877-26884).

The enzymes catalyzing the initial steps of guaianolide sesquiterpenes in chicory leading to the intermediate costunolide have been elucidated (Bouwmeester et al. Plant Physiol. 2002, 129: 134-144; Nguyen et al. J Biol Chem 2010, 285, 16588-16598; Cankar et al. FEBS Lett 2011, 585, 178-182; Liu et al. PLoS One 2011, 6, e23255; Ikezawa et al. J Biol Chem 2011, 286, 21601-21611) while the late steps in the STL production in chicory are poorly understood. One of the enzymes that has been subject to investigation is the group of germacrene A synthases, which are capable of converting farnesyl diphosphate (FPP) to germacrene A. In chicory the GAS family consists of four functional GAS genes, i.e. one GAS-long gene and three GAS-short genes. In most tissues, GAS-long gene expression outperforms GAS-short gene expression, especially in leaves where GAS-short expression was near to zero (Bouwmeester et al. Plant Physiol. 2002, 129: 134-144; Bogdanović et al. Industrial Crops & Products, 2019, 129: 253-260). Using an RNAi approach for targeting three out of the four functional GAS genes resulted in variable levels of reduction in sesquiterpene lactones in different lines, which did not reveal a clear correlation between RNAi-mediated gene suppression and STL levels, especially not in roots where GAS enzyme expression levels and STL levels suggest that other mechanisms or pathways may control STL levels (Bogdanović et al. 2019, GM Crops Food. October 31:1-13).

The development of crops with altered levels of STLs may lead to cost savings in inulin extraction from inulin producing (root) crops and the production of less bitter (leaf) crops thereby making these varieties more suitable for other markets. There is therefore a need in the art for plants having a reduced sesquiterpene lactone (STL) levels as well for methods for producing said plants.

Other compounds that are subject to the present invention are squalene and phenolic compounds.

Squalene is used for cosmetic applications and as an adjuvant in vaccines. Shark liver oil was used previously as the main source of squalene. Plants normally do not accumulate large quantities of squalene. However, some plant sources are enriched in squalene such as olive oil, soybean oil, rice, wheat germ, grape seed oil, peanut, corn, and amaranth (Alvarez-Suarez et al. International Journal of Agronomy 2018, 1687-8159). Olive oil is nowadays the only natural plant resource commercially exploited to obtain plant squalene. The content of squalene in olive oil ranges from 110-840 mg/100 g olive oil in different olive varieties (Beltran et al. Eur J Lipid Sci Tech, 2016, 118, 1250-1253). Biotechnological efforts have led to increased production of squalene in leaves of transgenic tobacco reaching a maximal yield of 670 ug/gFW upon overexpression of biosynthetic enzymes and targeting of these enzymes to the plastids (Jiang et al. Plant Biotechnol J 2018, 16, 1110-1124). The same biotechnological approach was employed to produce squalene in oilseed of Arabidopsis thaliana where accumulation of 227.30 μg/g seed for squalene was observed (Kempinski & Chappell, Plant Biotechnol J 2019, 17, 386-396).

Because of the interest for alternative resources of squalene, there is a need in the art for plants having increased squalene levels as well for methods for producing said plants.

Phenolic compounds are recognized for their health benefit effects and are the most important dietary antioxidants (Legrand et al. Front Plant Sci 2016, 7, 741).

Because of these health benefit and antioxidant activity of phenolic compounds, there is a need in the art for plants having increased levels of phenolic compounds as well for methods for producing said plants.

### SUMMARY OF THE INVENTION

The inventors have identified an unexpected decrease in STL production and unexpected increased levels of squalene and phenolic compounds upon reducing expression of GAS genes. The invention may be summarized in the following numbered embodiments

### Embodiment 1

Method for producing a plant having at least one of
a reduced sesquiterpene lactone (STL) level;
an increased squalene level; and
an increased level of a phenolic compound,
   as compared to a control plant, comprising the step of mutating one or more endogenous functional GAS-short genes in said plant resulting in a decreased or abolished expression of one or more functional GAS-short proteins and/or resulting in a decreased or abolished activity of one or more functional GAS-short proteins.

### Embodiment 2

Method according to embodiment 1, wherein the method comprises the step of mutating multiple, preferably all, endogenous functional GAS-short genes in said plant.

### Embodiment 3

Method according to any one of the preceding embodiments, wherein the method comprises a step of insertion, deletion or substitution of at least one nucleotide in the coding sequence of the one or more GAS-short genes, resulting in at least one of a decreased or abolished activity of the encoded GAS-short proteins.

### Embodiment 4

Method according to any one the preceding embodiments, wherein the method comprises a step of insertion, deletion or substitution of at least one nucleotide in at least one transcription regulatory sequence of the one or more GAS-short genes, resulting in decreased or abolished expression of the encoded GAS-short proteins.

### Embodiment 5

Method according to any one of the preceding embodiments, wherein the one or more endogenous functional GAS-short genes are homologues of any one of CiGAS-S1, CiGAS-52 and CiGAS-53.

### Embodiment 6

Method according to any one of the preceding embodiments, wherein the expression of said protein is impaired in at least any one of the leaves and the roots of said plant.

### Embodiment 7

Method according to any one of the preceding embodiments, wherein the method further comprises the step of regenerating said plant, and optionally further comprises at least one of the steps of:
inulin extraction;
squalene extraction; and
phenolic compound extraction,
   from said plant, preferably from the plant root.

### Embodiment 8

A nucleic acid comprising a GAS-short gene comprising one or more modifications, wherein said one or more modifications results in impaired expression of a functional GAS-short protein and/or results in impaired activity of the encoded functional GAS-short protein when said nucleic acid is present in a plant as compared to an identical nucleic acid not comprising said one or more modifications.

### Embodiment 9

A construct, vector or host cell comprising the nucleic acid of embodiment 8.

### Embodiment 10

A GAS-short protein having a modification that results in a decreased function as compared to an identical GAS-short protein not having said modification.

### Embodiment 11

A plant obtainable from a method according to any one of embodiments 1-7, or progeny thereof.

### Embodiment 12

A plant having at least one of:
a reduced sesquiterpene lactone (STL) level;
an increased squalene level; and
an increased level of a phenolic compound,
   as compared to a control plant, wherein said plant shows reduced expression and/or reduced activity of a functional GAS-short protein, or progeny thereof.

### Embodiment 13

Plant according to embodiment 11 or 12, wherein said plant comprises a nucleic acid of embodiment 8 or construct, vector or host cell according to embodiment 9, and/or wherein said plant expresses a modified GAS-short protein of embodiment 10, or progeny thereof.

### Embodiment 14

Method of producing at least one of inulin, squalene and a phenolic compound, wherein said method comprises the steps of
providing a plant according to any one of embodiments 11-13;
extracting at least one of inulin, squalene and a phenolic compound from said plant or plant part; and
optionally, purifying at least one of said inulin, squalene and a phenolic compound.

### Embodiment 15

Use of a nucleic acid of embodiment 8, construct, vector or host cell of embodiment 9 or modified GAS-short protein of embodiment 10 for at least one of
reducing the sesquiterpene lactone (STL) level;
increasing the squalene level; and
increasing the level of a phenolic compound,
   in a plant.

### Embodiment 16

Method for producing a plant having one or more mutated GAS-short genes, comprising the step of mutating one or more endogenous functional GAS-short genes in said plant resulting in a decreased or abolished expression of one or more functional GAS-short proteins and/or resulting in a decreased or abolished activity of one or more functional GAS-short proteins, and wherein the produced plant has at least one of
a reduced sesquiterpene lactone (STL) level;
an increased squalene level; and
an increased level of a phenolic compound,
   as compared to a control plant.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

It is clear for the skilled person that any methods and materials similar or equivalent to those described herein can be used for practicing the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 4th Edition, Cold Spring Harbor Laboratory Press, Cold Spring Harbor, N.Y., 2012; Ausubel et al. Current Protocols in Molecular Biology, John Wiley & Sons, New York, 1987 and periodic updates; the series Methods in Enzymology, Academic Press, San Diego and J M Walker, the series Methods in Molecular Biology, Springer Protocols.

The singular terms “a,” “an,” and “the” include plural referents unless the context clearly dictates otherwise. Thus, for example, reference to “a cell” includes a combination of two or more cells, and the like. The indefinite article “a” or “an” thus usually means “at least one”.

“Analogous to” in respect of a domain, sequence or position of a protein, in relation to an indicated domain, sequence or position of a reference protein, is to be understood herein as a domain, sequence or position that aligns to the indicated domain, sequence or position of the reference protein upon alignment of the protein to the reference protein using alignment algorithms as described herein, such as Needleman Wunsch. “Analogous to” in respect of a domain, sequence or position of a nucleic acid, in relation to an indicated domain, sequence or position of a reference nucleic acid, is to be understood herein as a domain, sequence or position that aligns to the indicated domain, sequence or position of the reference nucleic acid upon alignment of the nucleic acid to the reference nucleic acid using alignment algorithms as described herein, such as Needleman Wunsch.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ±(+ or −) 10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The term “impairing” is understood herein as at least one of decreasing and abolishing.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant cell. The protein of the invention may be at least one of a recombinant, synthetic or artificial protein.

“Plant” refers to either the whole plant or to parts of a plant, such as cells, protoplasts, calli, tissue, organs (e.g. embryos pollen, ovules, seeds, gametes, roots, leaves, flowers, flower buds, anthers, fruit, etc.) obtainable from the plant, as well as derivatives of any of these and progeny derived from such a plant by selfing or crossing. Non-limiting examples of plants include crop plants and cultivated plants, such as African eggplant, alliums, artichoke, asparagus, barley, bean, beet, bell pepper, bitter gourd, bladder cherry, bottle gourd, cabbage, canola, carrot, cassava, cauliflower, celery, chickpea, chicory, common bean, corn salad, cotton, cucumber, eggplant, endive, fennel, gherkin, grape, hot pepper, lettuce, lentil, lupin, maize, melon, oilseed rape, okra, parsley, parsnip, pea, pepino, pepper, potato, pumpkin, radish, rice, ridge gourd, rocket, rye, snake gourd, sorghum, soybean, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem, plant organ or an explant.

“Similar conditions” for culturing the plant/plant cells means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

A “homologue” may an orthologue (a gene in a different species evolved from a common ancestral gene) or a paralogue (a gene copy created by a duplication event within the same genome). A homologue of a gene comprising or consisting of a particular nucleotide sequence, is to be understood herein as comprising or consisting of a nucleotide sequence that has at least 50%, 60%, 70%, 80%, 90%, 95%, 96%, 97%, 98%, or 99% sequence identity to the particular sequence of said gene over its whole length, and preferably encodes a protein with the same functionality as encoded by said gene. A homologue of a protein having a particular amino acid sequence, is to be understood herein as an amino acid sequence that has at least 50%, 60%, 70%, 80%, 90%, 95%, 96%, 97%, 98%, or 99% sequence identity to the amino acid sequence of said protein over its whole length, and preferably has the same or similar functionality as said protein.

“Sequence identity” and “sequence similarity” can be determined by alignment of two amino acid or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWlN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “nucleic acid” or “polynucleotide” according to the present invention may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). The present invention contemplates any deoxyribonucleotide, ribonucleotide or nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA (optionally cDNA) or RNA, or a mixture thereof, and may exist permanently or transitionally in single-stranded or double-stranded form, including homoduplex, heteroduplex, and hybrid states. An “isolated nucleic acid” is used to refer to a nucleic acid which is no longer in its natural environment, for example in vitro or in a recombinant bacterial or plant cell. The nucleic acid of the invention may be at least one of a recombinant, synthetic or artificial nucleic acid.

The terms “nucleic acid construct”, “nucleic acid vector”, and “vector” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules. The vector backbone may for example be a binary or superbinary vector (see e.g. U.S. Pat. No. 5,591,616, US 2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid (e.g. comprising a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like. The construct or vector may be an “expression construct” or “expression vector” in case the vector comprises a sequence encoding for an RNA and/or protein, wherein said sequence is operably linked to appropriate regulatory regions, such as a promoter sequence.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, which is biologically active, e.g. a regulatory non-coding RNA or an RNA which is capable of being translated into a biologically active protein or peptide. Expression in relation to a protein or peptide is to be understood herein as the process of gene expression resulting in production of said protein or peptide.

The term “operably linked” refers to a linkage of polynucleotide elements in a functional relationship. A nucleic acid region is “operably linked” when it is placed into a functional relationship with another nucleic acid region. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation). A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

A “3′ UTR” or “3′ non-translated sequence” (also often referred to as 3′ untranslated region, or 3′end) refers to the nucleic acid sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

The term “cDNA” means complementary DNA. Complementary DNA is made by reverse transcribing RNA into a complementary DNA sequence. cDNA sequences thus correspond to RNA sequences that are expressed from genes. As mRNA sequences when expressed from the genome can undergo splicing, i.e. introns are spliced out of the mRNA and exons are joined together, before being translated in the cytoplasm into proteins, it is understood that expression of a cDNA means expression of the mRNA that encodes for the cDNA. The cDNA sequence thus may not be identical to the genomic DNA sequence to which it corresponds as cDNA may comprise only the complete open reading frame, consisting of the joined exons, for a protein, whereas the genomic DNA may comprise exons interspersed by intron sequences. Impairment of expression a protein by genetic modification of a gene encoding the protein may thus not only relate to modifying the sequences encoding the protein, but may also involve mutating intronic sequences of the genomic DNA and/or other gene regulatory sequences of that gene, as long as it results in the impairment of gene expression.

The term “regeneration” is herein defined as the formation of a new plant, new tissue and/or a new organ from a single plant cell, a callus, an explant, a tissue or from an organ. The regeneration pathway can be somatic embryogenesis or organogenesis. Somatic embryogenesis is understood herein as the formation of somatic embryos, which can be grown to regenerate whole plants. Organogenesis is understood herein as the formation of new organs from (undifferentiated) cells. Preferably, the regeneration is at least one of ectopic apical meristem formation, shoot regeneration and root regeneration. The regeneration as defined herein can preferably concern at least de novo shoot formation. For example, regeneration can be the regeneration of a(n) (elongated) hypocotyl explant towards a(n) (inflorescence) shoot. Regeneration may further include the formation of a new plant from a single plant cell or from e.g. a callus, an explant, a tissue or an organ. The regeneration process can occur directly from parental tissues or indirectly, e.g. via the formation of a callus.

The term “conditions that allow for regeneration” is herein understood as an environment wherein a plant cell or tissue can regenerate. Such conditions include at minimum a suitable temperature (i.e. between 0° C.-60° C.), nutrition and day/night rhythm. Furthermore, “optimal conditions that allow for regeneration” are those environmental conditions that allow for a maximum regeneration of the plant cells.

The term “wild type” as used in the context of the present invention in combination with a protein or nucleic acid means that said protein or nucleic acid consists of an amino acid or nucleotide sequence, respectively, that occurs as a whole in nature and can be isolated from organisms in nature as such, e.g. is not the result of modification techniques such as targeted or random mutagenesis or the like. A wild type protein is expressed in at least a particular cell type, in a particular developmental stage under particular environmental conditions, e.g. as it occurs in nature.

The term “endogenous” as used in the context of the present invention in combination with a protein or nucleic acid (e.g. gene) means that said protein or nucleic acid originates from the plant in which it is still contained. Often an endogenous protein or nucleic acid will be present in its normal genetic context in the plant. In the present invention, an endogenous protein or nucleic acid may be modified in situ (in the plant or plant cell) using standard molecular biology methods, e.g. gene silencing, random mutagenesis or targeted mutagenesis.

The term “GAS” protein or gene refers to a germacrene A synthase protein or gene encoding the same, wherein said protein has germacrene A synthase activity. Germacrene A synthase activity is the ability to convert farnesyl diphosphate to germacrene A.

Unless stated otherwise the term “GAS protein” includes at least one of a GAS-short protein and a GAS-long protein. A GAS-short protein is, or is a homologue of, a protein having an amino acid sequence of any one of SEQ ID NO: 1-6 and/or encoded by any one of SEQ ID NO: 7-12. Preferably, the GAS-short protein is a wild type protein.

A GAS-short gene is a gene encoding a germacrene A synthase protein and preferably is a gene comprising a coding sequence having at least 70%, 75%, 80%, 85%, 90%, 91%, 92%, 93%, 94%, 95%, 96%, 97%, 98%, 99% or 100% identity to, any one of SEQ ID NO: 7-12 and/or encoding a protein of any one of SEQ ID NO: 1-6, or homologue thereof.

Preferably, a GAS-shod gene comprises a coding sequence having at least 70%, 75%, 80%, 85%, 90%, 91%, 92%, 93%, 94%, 95%, 96%, 97%, 98%, 99% or 100% identity to SEQ ID NO: 7.

Preferably, a GAS-shod gene comprises a coding sequence having at least 70%, 75%, 80%, 85%, 90%, 91%, 92%, 93%, 94%, 95%, 96%, 97%, 98%, 99% or 100% identity to SEQ ID NO: 8.

Preferably, a GAS-shod gene comprises a coding sequence having at least 70%, 75%, 80%, 85%, 90%, 91%, 92%, 93%, 94%, 95%, 96%, 97%, 98%, 99% or 100% identity to SEQ ID NO: 9.

Preferably, a GAS-shod gene comprises a coding sequence having at least 70%, 75%, 80%, 85%, 90%, 91%, 92%, 93%, 94%, 95%, 96%, 97%, 98%, 99% or 100% identity to SEQ ID NO: 10.

Preferably, a GAS-shod gene comprises a coding sequence having at least 70%, 75%, 80%, 85%, 90%, 91%, 92%, 93%, 94%, 95%, 96%, 97%, 98%, 99% or 100% identity to SEQ ID NO: 11.

Preferably, a GAS-shod gene comprises a coding sequence having at least 70%, 75%, 80%, 85%, 90%, 91%, 92%, 93%, 94%, 95%, 96%, 97%, 98%, 99% or 100% identity to SEQ ID NO: 12.

Preferably, a GAS-shod gene encodes a protein having at least 70%, 75%, 80%, 85%, 90%, 91%, 92%, 93%, 94%, 95%, 96%, 97%, 98%, 99% or 100% identity to SEQ ID NO: 1.

Preferably, a GAS-shod gene encodes a protein having at least 70%, 75%, 80%, 85%, 90%, 91%, 92%, 93%, 94%, 95%, 96%, 97%, 98%, 99% or 100% identity to SEQ ID NO: 2.

Preferably, a GAS-shod gene encodes a protein having at least 70%, 75%, 80%, 85%, 90%, 91%, 92%, 93%, 94%, 95%, 96%, 97%, 98%, 99% or 100% identity to SEQ ID NO: 3.

Preferably, a GAS-shod gene encodes a protein having at least 70%, 75%, 80%, 85%, 90%, 91%, 92%, 93%, 94%, 95%, 96%, 97%, 98%, 99% or 100% identity to SEQ ID NO: 4.

Preferably, a GAS-shod gene encodes a protein having at least 70%, 75%, 80%, 85%, 90%, 91%, 92%, 93%, 94%, 95%, 96%, 97%, 98%, 99% or 100% identity to SEQ ID NO: 5.

Preferably, a GAS-shod gene encodes a protein having at least 70%, 75%, 80%, 85%, 90%, 91%, 92%, 93%, 94%, 95%, 96%, 97%, 98%, 99% or 100% identity to SEQ ID NO: 6.

Preferably, the GAS-shod protein has a 40 amino acid long N-terminal domain that has at least 70%, 75%, 80%, 85%, 90%, 91%, 92%, 93%, 94%, 95%, 96%, 97%, 98%, 99% or 100% identity to SEQ ID NO: 15 over its whole length. Preferably, the GAS-shod protein lacks the N-terminal domain of a GAS-long protein, wherein said N-terminal domain of the GAS-long protein is preferably the 40 amino acid long sequence having at least 80%, 85%, 90%, 91%, 92%, 93%, 94%, 95%, 96%, 97%, 98%, 99% or 100% identity to SEQ ID NO: 16 over its whole length. Preferably, a GAS-shod gene encodes for a protein of at most about 580, 575 or 570 amino acids. Preferably, a GAS-shod gene is a gene within the phylogenetic clade II as described in Nguyen et al. Biochem Biophys Res Commun. 2016 Oct. 28; 479(4):622-627; in particular see FIG. 3 thereof). Examples of GAS-shod proteins are proteins having the amino acid sequence of any one of SEQ ID NO: 1 and 2 (CiGAS-S1), SEQ ID NO: 3 and 4 (CiGAS-52), SEQ ID NO: 5 and 6 (CiGAS-53), and/or sequences having NCBI accession number of any one of KM066977, DQ447636, AF489964, AF489965, AF498000, JQ255377, DQ016667, EU327785, GU176380, DQ186657, JN383985, KC441526, JF819848, KC145534 and KJ194511.

A GAS-long gene is a gene encoding a germacrene A synthase and preferably is a gene comprising a coding sequence having at least 70%, 75%, 80%, 85%, 90%, 91%, 92%, 93%, 94%, 95%, 96%, 97%, 98%, 99% or 100% identity to SEQ ID NO: 14 and/or encoding a protein of SEQ ID NO: 13, or homologue thereof. A GAS-long protein is, or is a homologue of, a protein having an amino acid sequence of SEQ ID NO: 13 and/or encoded by SEQ ID NO: 14. Preferably, the GAS-long protein is a wild type protein. Preferably, the GAS-long protein has a 40 amino acid long N-terminal domain that has at least 70%, 75%, 80%, 85%, 90%, 91%, 92%, 93%, 94%, 95%, 96%, 97%, 98%, 99% or 100% identity to SEQ ID NO: 16 over its whole length. Preferably, a GAS-long gene is a gene within clade I as described in Nguyen et al., Biochem Biophys Res Commun. 2016 Oct. 28; 479(4):622-627; in particular see FIG. 3 thereof). Examples of GAS-long proteins are proteins having the amino acid sequence of SEQ ID NO: 13 (CiGAS-L1) and/or sequence having NCBI accession numbers of any one of KM066976, KU234689, AF497999 and AY082672.

“Mutagenesis” and/or “modification of a gene or nucleic acid” may be random mutagenesis or targeted mutagenesis resulting in one or more altered or mutated nucleic acid(s). Random mutagenesis may be, but is not limited to, chemical mutagenesis and gamma radiation. Non-limiting examples of chemical mutagenesis include, but are not limited to, EMS (ethyl methanesulfonate), MMS (methyl methanesulfonate), NaN3 (sodium azide) D), ENU (N-ethyl-N-nitrosourea), AzaC (azacytidine) and NQO (4-nitroquinoline 1-oxide). Optionally, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines with a modified gene as defined herein. TILLING uses traditional chemical mutagenesis (e.g. EMS mutagenesis) followed by high-throughput screening for mutations. Thus, plants, seeds and tissues comprising a gene having one or more of the desired mutations may be obtained using TILLING. Targeted mutagenesis is mutagenesis that can be designed to alter a specific nucleotides or nucleic acid sequence, such as but not limited to, oligo-directed mutagenesis, RNA-guided endonucleases (e.g. CRISPR-technology), meganucleases, TALENs or Zinc finger technology.

A “phenolic compound” has an ordinary meaning known to the person skilled in the art. The phenolic compound is preferably a plant, or plant-derived, phenolic compound. Phenolic compounds are a large class of plant secondary metabolites, showing a diversity of structures, from rather simple structures, e.g. phenolic acids, through polyphenols such as flavonoids, that comprise several groups, to polymeric compounds based on these different classes (Cheynier V, Phytochemistry Reviews, 2012 volume 11, pages 153-177). Phenolic compounds contain benzene rings, preferably with one or more hydroxyl substituents, and range from simple phenolic molecules to highly polymerized compounds. The effects of plant phenolic compounds on human nutrition are e.g. reviewed in Lin D. et al, Molecules. 2016 October; 21(10): 1374. A particularly preferred phenolic compound is selected from the group consisting of 3,5-dicaffeoylquinic acid, chlorogenic acid and chicoric acid.

A “control plant” as referred to herein is a plant of the same species and preferably same genetic background as the plant that is, or is a progeny of, a plant (or “putative test plant” or “test plant”) that has been subjected to a method as taught herein, i.e. a method for at least one of reducing STL production, increasing squalene level and increasing the level of a phenolic compound. Alternatively or in addition, a “control” plant as referred to herein is a plant of the same species and preferably same genetic background as the plant of the invention, with the exception that the control plant does not comprise one or more mutated GAS-short genes as defined herein. The control plant preferably comprises an endogenous GAS-short gene and expresses the encoded GAS-short protein. The control plant preferably produces STL. In addition or alternatively, the control plant may accumulate a limited amount of squalene, such as, but not limited to, a low or even negligible level of squalene. In addition or alternatively, the control plant may accumulate limited levels of a phenolic compound, such as, but not limited to a low or even negligible level of a phenolic compound. The control plant may produce STL, a limited amount of squalene and a limited level of a phenolic compound, or a combination thereof depending whether such plant may serve as a control for a plant having reduced STL production, increased squalene levels, increased phenolic compounds levels, or a combination thereof, respectively. Preferably, the control plant only differs from the putative test plant in the protein, nucleic acid and/or vector or construct of the invention. Preferably the control plant is grown under the same conditions as the test plant comprising the protein and/or nucleic acid of the invention.

“A limited level” or “limited amount” of either squalene or a phenolic compound is understood herein as a level that can be further increased, e.g. upon genetic modification of the plant cell.

“Reduced STL levels” or “reduced STL production” refers to a decrease in sesquiterpene lactones (STL) level of a plant, plant tissue or plant cell compared to a suitable control plant. Preferably, a plant, plant tissue or plant cell having decreased STL levels is a plant, plant tissue or plant cell comprising a reduction of at least 1%, 2%, 5%, 10%, 15%, 20%, 30%, 50%, 70%, 80%, 90%, or even 100% in level of one or more STLs as compared to the control plant. STLs are compounds known in the art, such as, but are not limited to, lactucin, lactucopicrin, 8-deoxy lactucin, and oxalates thereof, e.g., lactucin 15-oxalate, lactucopicrin 15-oxalate and 8-deoxy lactucin 15-oxalate. Preferably, the reduction in STL levels is a reduction of all STLs of said plant cell, plant or plant tissue.

“Enhanced squalene level(s)” or “increased squalene” refers to an increase in squalene level(s) or amount(s) in a plant, plant tissue or plant cell compared to a suitable control plant. Preferably, a plant, plant tissue or plant cell having increased squalene levels is a plant, plant tissue or plant cell comprising an increase of at least 1%, 2%, 5%, 10%, 15%, 20%, 30%, 50%, 70%, 80%, 90%, 100%, 200%, 500%, 700% or even 1000% in squalene levels as compared to the control plant. Preferably, a plant, plant tissue or plant cell having increased squalene levels is a plant, plant tissue or plant cell having a fold increase in squalene levels of at least about 1.2, 1.5, 2, 3, 5, 10, 20, 50, 60, 100, 200, 500 or 1000-fold as compared to the control plant.

“Enhanced phenolic compound level(s)” or “increased phenolic compound(s)” refers to an increase in phenolic compound level(s) or amount(s) of a plant, plant tissue or plant cell compared to a suitable control plant. Preferably, a plant, plant tissue or plant cell having increased phenolic compound levels is a plant, plant tissue or plant cell comprising an increase of at least 1%, 2%, 5%, 10%, 15%, 20%, 30%, 50%, 70%, 80%, 90%, 100%, 200%, 500%, 700% or even 1000% in phenolic compound levels as compared to the control plant. Preferably, a plant, plant tissue or plant cell having increased phenolic compound levels is a plant, plant tissue or plant cell having a fold increase in phenolic compound levels of at least about 1.2, 1.5, 2, 3, 5, 10, 20, 50, 60, 100, 200, 500 or 1000-fold as compared to the control plant.

The term “impairing the expression of a gene” as used herein, refers to a situation where the level of protein or RNA expressed from said gene in a modified plant or plant cell is reduced compared to the level of said RNA or protein that is expressed in a suitable control plant or plant cell (e.g., a wild type plant or plant cell). Preferably, expression of a gene is impaired when the level of RNA or protein expressed from said gene in a plant or plant cell is at least 1%, 2%, 5%, 10%, 15%, 20%, 30%, 50%, 70%, 80%, 90%, or even 100% lower than the level of RNA or protein expressed from said gene in the control plant. Alternatively, expression of a gene is impaired when the level of RNA or protein expressed from said gene in a modified plant or plant cell is statistically significantly lower than the level of RNA or protein that is expressed from the control plant.

The term “impairing the expression of a protein” as used herein, refers to a situation where the level of said protein in a modified plant or plant cell is reduced compared to the level of said protein produced in a suitable control plant or plant cell (e.g., a wild type plant or plant cell). Preferably, expression of a protein is impaired when the level of said protein produced in a plant or plant cell is at least 1%, 2%, 5%, 10%, 15%, 20%, 30%, 50%, 70%, 80%, 90%, or even 100% lower than the level of said protein that is produced in the control plant. Alternatively, expression of a protein is impaired when the level of said protein produced in a plant or plant cell is statistically significantly lower than the level of protein that is produced in the control plant.

The term “reduced activity of a protein” as used herein refers to a situation wherein the natural activity of a protein, such as for example its ability to bind to a promoter element, to bind to a receptor, to catalyse an enzymatic reaction, to regulate gene expression, etc, is altered or reduced or blocked or inhibited, for instance due to a modification in structure, as compared to the activity of the same protein albeit without said modification, preferably in a plant or plant cell. Preferably, the activity of a modified protein may be considered to be impaired when the activity of said modified protein produced in a plant or plant cell is at least 1%, 2%, 5%, 10%, 15%, 20%, 30%, 40%, 50%, 70%, 80%, 90%, or even 100% lower than the activity of the same protein without said modification as produced in a control plant. The skilled person will readily be capable of establishing whether or not activity of a protein is impaired. Preferably the protein is a GAS enzyme and the activity is the ability to convert farnesyl diphosphate (FPP) to germacrene A. Preferably, the activity of a functional GAS protein is impaired. A functional GAS protein is to be understood herein as a protein having germacrene A synthase activity, i.e. being capable of conferring farnesyl diphosphate (FPP) to germacrene A, preferably when present in a plant, even more preferably when present in chicory. Optionally, a functional GAS protein has activity comparable to a protein having any one of SEQ ID NO: 1-6, preferably having at least 20%, 30%, 40%, 50%, 70%, 80%, 90%, or even 100% of the activity of a protein having any one of SEQ ID NO: 1-6. Preferably, a functional GAS-short protein has activity comparable to a protein having any one of SEQ ID NO: 3 and 4, preferably having at least 20%, 30%, 40%, 50%, 70%, 80%, 90%, or even 100% of the activity of a protein having any one of SEQ ID NO: 3 and 4. Preferably, a functional GAS-long protein has activity comparable to a protein having SEQ ID NO: 13, preferably having at least 20%, 30%, 40%, 50%, 70%, 80%, 90%, or even 100% of the activity of SEQ ID NO: 13.

### DETAILED DESCRIPTION OF THE INVENTION

The inventors unexpectedly found a significant reduction in STL levels in both plant leaf and plant root by reducing expression of at least one more of the functional GAS-short genes, and even near to complete reduction by reducing expression of all functional GAS-short genes. This is unexpected as at least for root the art suggests other mechanisms or pathways than GAS enzymes to control STL levels, and because especially in leaf, GAS-long is predominantly expressed, while showing a very low GAS-short expression (Bogdanovic et al. Industrial Crops and Products 2019, 129, 253-260). The art therefore suggest that the GAS-long gene and not the GAS-short variants are responsible for STL accumulation. Generating crops with reduced STL synthesis are desired for instance in order to reduce bitterness and for further processing such as inulin extraction. Due to its self-incompatibility and because of the fact that their genomes comprise multiple GAS genes, mutation breeding of GAS enzymes in chicory and in other Asteraceae crops such as lettuce is seriously hampered.

Further, the inventors unexpectedly found a significant increase in squalene levels in plant root by reducing expression of at least one or more of the functional GAS-short genes, and no significant further increase in squalene levels were observed when additionally reducing expression of the functional GAS-long gene. This is unexpected since the art suggests that increased levels of terpenes leads to feedback regulation of its biosynthetic enzymes in the mevalonate pathway. It is unexpected that knocking out the GAS-short variants, while not affecting the GAS-long gene is sufficient for the squalene levels to peak. Generating crops with increased squalene levels is desired for instance for extracting and using said squalene for industrial applications of the squalene such as for cosmetic applications or as an adjuvant in vaccines.

The inventors now found a way of producing squalene that differs in that it does not require overexpression of endogenous of heterologous enzymes via transgenic approaches, but instead by knocking out one or more endogenous GAS genes. The content of squalene in chicory roots can optionally be further enhanced, e.g. using further gene-editing and/or via approaches documented for tobacco.

In addition, the inventors unexpectedly found a significant increase in phenolic compound levels in plant leaf and root by reducing expression of at least one more of the functional GAS-short genes, and no significant further increase in phenolic compound levels were observed when additionally reducing expression of the functional GAS-long gene. This is unexpected since phenolic compounds are biosynthesized by a pathway that is unrelated to the terpene biosynthetic pathways to which the GAS proteins belong. Further, it is unexpected that knocking out the GAS-short variants, while not affecting the GAS-long gene is sufficient for the phenolic compound levels to peak. Generating crops with increased phenolic compound levels are desired because of they are known in the art for their beneficial health effects.

In an aspect, the invention encompasses a nucleic acid comprising a GAS gene that has one or more modifications resulting in impaired expression and/or impaired activity of a functional GAS protein. Preferably, said GAS gene is a GAS-short gene and said functional GAS protein is a functional GAS-short protein. Therefore, the invention encompasses a nucleic acid comprising a GAS-short gene that has one or more modifications resulting in impaired expression and/or impaired activity of a functional GAS-short protein. Optionally, the invention encompasses a nucleic acid comprising one or more, preferably two or three, GAS-short genes each having one or more modifications resulting in impaired expression and/or impaired activity of a functional GAS-short protein. Alternatively or in addition, the invention encompasses a nucleic acid comprising a GAS-long gene that has one or more modifications resulting in impaired expression and/or impaired activity of a functional GAS-long protein.

This GAS gene of the invention is also denominated herein as a modified GAS gene, i.e. a modified GAS-short gene or modified GAS-long gene. Preferably, the modified GAS gene of the invention is derived from a wild type and/or an endogenous GAS gene by genetic modification. Said wild type and/or endogenous GAS gene is preferably a plant GAS gene. The one or more modifications of the wild type or endogenous GAS gene may result in impaired expression and/or impaired activity of the functional GAS protein encoded by said modified GAS gene as compared to the unmodified gene. The modified GAS gene of the invention preferably is a modified endogenous GAS gene, wherein the modified GAS gene shows at least one of a reduced or abolished expression and reduced or abolished activity of the encoded GAS protein when present in a plant as compared to the endogenous GAS gene in a control plant.

Optionally, the modified gene is obtained from said endogenous gene by deletion, insertion and/or substitution of at least one nucleotide, wherein said deletion, insertion and/or substitution results in a gene with impaired or abolished expression and/or decreased or abolished activity of the encoded GAS protein. Said modified gene may be obtained via random or targeted mutagenesis. Such modification may be within the coding sequence of said gene, resulting in a modified protein which is less functional as compared to the protein encoded by the unmodified GAS gene or which is a dysfunctional protein, wherein a dysfunctional protein is to be understood as a protein not being capable of fulfilling the function of the protein encoded by the unmodified GAS gene. Such modification may hence result in a protein having a decreased or abolished activity. Optionally, the modification is a frame shift mutation and/or introduces an early stop which results in a truncated protein which has a reduced function and may be dysfunctional. Preferably said modification is in exon 4 of the GAS gene, or any domain analogous to exon 4 of the GAS genes exemplified herein, preferably resulting one or more amino acid deletions or one or more amino acid substitutions, wherein preferably the one or more nucleotide deletions result in a frame shift. Optionally, the modified GAS gene is obtained by using a CRISPR complex comprising a CRISPR endonuclease and a guide RNA for targeting the complex to a sequence that is, or is homologous to, at least one of SEQ ID NO: 22, SEQ ID NO: 23 and SEQ ID NO: 24, preferably at least one of SEQ ID NO: 22 and SEQ ID NO: 23, even more preferably SEQ ID NO: 22. For instance, the complex may be a Cpf1-crRNA complex or a Cas9-crRNA-tracrRNA complex, wherein in the latter case the crRNA and tracrRNA may be a separate molecules (dual guide RNA or dgRNA) or covalently linked molecules (single guide RNA or dgRNA). The person skilled in the art knows how to design such CRISPR complexes, including a CRIPSR endonuclease and guide RNA for targeting the GAS genes as defined herein. For instance, referred is to PCT/EP2019/079950, PCT/EP2019/068839 and WO2018/115390, which are incorporated herein by reference.

The CRISPR complex may be introduced in the cell(s) comprising the gene to be modified using a ribonucleoprotein (RNP, i.e. a CRISPR endonuclease protein complexed with a guide RNA) or one or more vectors encoding the components of the RNP. In case of an RNP based transfection, the RNA backbone of the sgRNA or dgRNA of the RNP preferably comprises modifications such as phosphorothioate and/or 2′-O-methyl RNA moieties, preferably at either end of the RNA backbones, to protect the RNAs from nuclease degradation. Optionally, multiple complexes are used to target multiple different GAS genes in a cell, and/or targeting the same GAS gene at different positions. For instance, a vector encoding a CRISPR nuclease (e.g. Cas9 having the sequence of SEQ ID NO: 61 encoded by SEQ ID NO: 62) and a vector encoding one or more guides may be used. Preferably, the CRISPR nuclease open reading frame within the vector is operably linked to a promoter suitable for protein expression in plants, e.g. Arabidopsis ubiquitin promoter of SEQ ID NO: 66. Preferably, the guide RNA encoding sequences are operably linked to a promoter suitable for small RNA expression in plants, e.g. an Arabidopsis U6 promoter of SEQ ID NO: 60. In case a Cas9 molecule is used, the guide RNA may be a single guide comprising an about 20 nucleotides long gene specific sequence and a scaffold at the 3′ end of the gene specific sequence, wherein said scaffold optionally has the sequence of SEQ ID NO: 63. In combination with a Cas9, also a dual guide RNA may be used optionally comprising a crRNA having the sequence of SEQ ID NO: 64 appended to the 3′-end of an about 20 nucleotides long gene specific sequence and a tracrRNA of the dgRNA may have the sequence of SEQ ID NO: 65. Optionally, the modified GAS gene is obtained using at least one CRISPR complex comprising a sgRNA having the sequence of SEQ ID NO: 17, 18 or 19, or construct encoding the same, which are also encompassed by the present invention. Preferably, the modified GAS-short gene is obtained by using a CRISPR endonuclease targeted to a sequence that is, or is homologous to, any one of SEQ ID NO: 22 and SEQ ID NO: 23, e.g. using at least one CRISPR complex comprising a sgRNA having the sequence of SEQ ID NO: 17 or 18, or constructs encoding the same such as SEQ ID NO: 20 for targeting SEQ ID NO: 17. Preferably, the modified GAS-long gene is obtained by using a CRISPR endonuclease targeted to a sequence that is, or is homologous to, SEQ ID NO: 24, e.g. using a CRISPR complex comprising a sgRNA having the sequence of SEQ ID NO: 19, or constructs encoding the same.

In case multiple guide RNAs are used in order to target multiple GAS genes and/or to target a GAS gene at multiple locations within a cell, a construct encoding multiple guides can be used, wherein the encoding sequences are preferably operably linked to a single promoter sequence suitable for inducing expression in the (host) cell, preferably a plant cell, such as an Arabidopsis U6 promoter e.g., the promoter of SEQ ID NO: 60, and the encoded sequences may be separated by tRNA sequences for optimal splicing, wherein a tRNA sequence may be the sequence as defined herein by SEQ ID NO: 59. An exemplary construct encoding guide RNA sequences targeting the multiple GAS genes (Gas-S1, GAS-S2, GAS-S3 and GAS-L1) for use in combination with a Cas9 endonuclease is defined herein by SEQ ID NO: 21.

Genetic modification of an endogenous GAS gene resulting in reduced or abolished expression and/or activity of the encoded protein results in at least one of decreased STL levels, increased squalene levels and increased phenolic compound levels, preferably a combination thereof, preferably the combination of all three, as compared to a control plant expressing the protein encoded by the unmodified gene, when grown under similar conditions. Moreover, expression of a modified and/or truncated protein in a plant encoded by a modified GAS gene of the invention, preferably in the absence of expression of the protein encoded by the unmodified gene, e.g. the unmodified endogenous gene, results in at least one of decreased STL levels, increased squalene levels and increased phenolic compound levels, preferably a combination thereof, preferably the combination of all three, as compared to a control plant expressing the protein encoded by the unmodified gene.

Preferably, STL levels of a plant comprising the modified endogenous GAS gene of the invention are reduced as compared to the STL levels of a control plant not comprising said modification, when grown under similar conditions. Preferably, squalene levels of a plant comprising the modified endogenous GAS gene of the invention are increased as compared to the squalene levels of a control plant not comprising said modification, when grown under similar conditions. Preferably, phenolic compound levels of a plant comprising the modified endogenous GAS gene of the invention are reduced as compared to the phenolic compound levels of a control plant not comprising said modification, when grown under similar conditions. Preferably, STL levels are reduced, squalene levels are increased and phenolic compound levels are increased in a plant comprising the modified endogenous GAS gene of the invention as compared to respectively the STL levels, the squalene levels and the phenolic compound levels of a control plant not comprising said modification, when grown under similar conditions. Optionally, a plant comprising two or more modified endogenous GAS genes, preferably GAS-short genes, of the invention shows at least one of at least one of reduced STL levels, increased squalene levels and increased phenolic compound levels, preferably a combination thereof, preferably a combination of all three, as compared to a control plant comprising the unmodified endogenous counterparts.

Optionally, the modification of the coding sequence results in a frame shift, preferably said frame shift mutation being in exon 4 of the GAS gene as defined herein, resulting in a dysfunctional encoded GAS protein in the cell. Optionally, the modification of the coding sequence is the deletion of all or most of the nucleotides of the sequence encoding the GAS protein, resulting in an absence of the encoded GAS protein in the cell. Optionally, the modification of the coding sequence results in the expression of an aberrant mRNA molecule that e.g. is no longer recognized by the translational machinery and degraded prior to translation.

In addition or alternatively, such modification may be in a regulatory sequence, such as the promoter sequence, resulting in impaired or abolished expression of a functional protein.

In addition or alternatively, the modified GAS gene may comprise one or more epigenetic modifications that reduce or silence gene expression.

Preferably, the unmodified GAS gene encodes for a protein that is, or is a homologue of, a protein having an amino acid sequence of any one of SEQ ID NO: 1-6 and 13. Preferably, the unmodified GAS-short gene encodes for a protein that is, or is a homologue of, a protein having an amino acid sequence of any one of SEQ ID NO: 1-6. Preferably, the unmodified GAS-long gene encodes fora protein that is, or is a homologue of, a protein having an amino acid sequence of SEQ ID NO: 13. Preferably, the modified GAS gene is derived by genetic modification from a GAS-short gene that comprises a coding sequence that has at least 70%, 75%, 80%, 85%, 90%, 91%, 92%, 93%, 94%, 95%, 96%, 97%, 98%, 99% or 100% identity to any one of SEQ ID NO: 7-12 over its whole length. Preferably, the modified GAS-short gene is derived by genetic modification from a GAS-short gene that is, or is a homologue of, a GAS-short gene comprising a coding sequence of any one of SEQ ID NO: 7-12, preferably of SEQ ID NO: 9 or 10. Preferably, the modified GAS-long gene is derived by genetic modification from a GAS-long gene that is, or is a homologue of, a GAS-long gene that comprises a coding sequence that has at least 70%, 75%, 80%, 85%, 90%, 91%, 92%, 93%, 94%, 95%, 96%, 97%, 98%, 99% or 100% identity to SEQ ID NO: 14 over its whole length.

Preferably, the modified GAS gene of the invention shows at least 50%, 60%, 70%, 80%, 90%, 95%, 96%, 97%, 98%, or 99% sequence identity to the GAS gene were it is derived from, wherein the latter may be an endogenous GAS gene as defined herein.

Preferably, expression and/or activity of the GAS protein of the modified GAS gene is impaired at least in the roots of a plant and/or at least in plant root cells. Inulin may be extracted from said roots and/or root cells, preferably resulting in reduced effort and/or cost for inulin extraction from said roots or root cells. Optionally, expression and/or activity of the GAS protein is impaired in the leaves of said plant and/or in plant leaf cells, preferably resulting in less bitter taste of said leaves and/or leaf cells.

In an embodiment, the phenotype of the plant as taught herein is not altered as compared to a control plant, with the exception of said plant having at least one of reduced STL levels, increased squalene levels and increased phenolic compound levels, preferably a combination thereof, preferably a combination of all three, as compared to said control plant. For instance, yield, root size, leaf size, reproduction, flowering, growth, development, color etc. is not affected in plants subjected to the methods according to the invention compared to a control plant or wild type plant, preferably of the same species.

The nucleic acid of the invention may be located in an expression construct or within the genome of a cell, preferably a plant cell. The invention therefore also provides for a construct or vector comprising the nucleic acid as defined herein and/or encoding the protein of the invention. The construct may be an expression construct for expressing the modified GAS gene of the invention and/or expression of the modified GAS protein of the invention.

Preferably, within a construct or vector of the invention, the nucleic acid is operably linked to one or more transcription regulatory elements for expression in a cell such as a 5′ UTR and 3′ UTR, preferably at least to a promoter for expression in a plant cell. Hence preferably, the nucleic acid construct comprises a nucleic acid as defined herein that is operably linked to a promoter for expression in a cell, such as a bacterial cell or a plant cell. Preferably, the nucleic acid according to the invention is operably linked to a promoter for expression in a plant cell. The promoter for expression in plant cells can be a constitutive promoter, an inducible promoter or a tissue specific promoter. Preferably, the promoter is a constitutive promoter. The promoter for expression in plant cells is herein understood as a promoter that is active in plants or plant cells, i.e. the promoter has the general capability to control transcription within a plant or plant cell. Preferably, the promoter is active in at least the root cells of a plant. Optionally, the promoter is only active in the root cells of a plant. In another embodiment, the promoter is active in at least the leaf cells of a plant. Optionally the promoter is only active in the leaf cells of a plant.

Preferably, the modified gene of the invention is capable of at least one of reducing or abolishing STL levels, increasing or inducing squalene levels and increasing or inducing phenolic compound levels, preferably a combination thereof, preferably a combination of all three, of a plant when present in said plant, as compared to a control plant comprising the unmodified counterpart, wherein the unmodified counterpart preferably is an endogenous GAS gene. Preferably, the plant comprising the modified GAS gene of the invention does not comprise the unmodified counterpart. Preferably, STL levels of a plant comprising the nucleic acid of the invention, also indicated herein as the test plant, is reduced as compared to a control plant. Preferably, squalene levels of a plant comprising the nucleic acid of the invention, also indicated herein as the test plant, is increased as compared to a control plant. Preferably, phenolic compound levels of a plant comprising the nucleic acid of the invention, also indicated herein as the test plant, is increased as compared to a control plant. Preferably, STL levels are reduced and squalene and phenolic compound levels are increased in a plant comprising the nucleic acid of the invention, also indicated herein as the test plant, as compared to a control plant. Preferably, the test plant comprises a modified endogenous GAS gene as defined herein, and the control plant comprises the unmodified endogenous GAS gene. Optionally, the test plant comprises two or more modified endogenous GAS genes as defined herein. For instance, chicory comprises four GAS genes (three GAS-short genes and one GAS-long gene), each having two alleles. Optionally, two, three, four, five, six, seven or all eight of these GAS alleles in a chicory plant are modified to impair expression of a functional GAS protein. Preferably, two, three, four, five or all six of the GAS-short alleles in a chicory plant are modified to impair expression of a functional GAS-short protein, which results in a at least one of a strong reduction of STL levels, a strong increase in squalene levels and a strong increase in phenolic compound levels, or a combination thereof, preferably a combination of all three, in said plant, as compared to a control chicory plant that comprises the unmodified counterparts of these alleles.

Preferably, at least two alleles of at least one of CiGAS-52 and CiGAS-S1, or homologue thereof, are modified to impair expression of at least one of a functional CiGAS-52 and CiGAS-S1 protein, or homologue thereof.

The nucleic acid of the invention may be DNA, cDNA or RNA. The nucleic acid can be transiently introduced into the plant cell, e.g. by transient transfection of a plasmid, optionally in combination with impairing or reducing expression, knocking out and/or silencing (e.g. by RNAi) one or more endogenous GAS genes of said plant cell. Alternatively or in addition, the nucleic acid can be stably present in the genome of the plant cell. As a non-limiting example, the nucleic acid may be stably integrated into the genome of the plant cell. Alternatively or in addition, the nucleic acid can be a modified wild type nucleic acid, e.g. a wild type and/or endogenous nucleic acid that is modified to have reduced or absence of GAS expression or is modified to encode the protein of the invention. In this embodiment, the nucleic acid of the invention is preferably DNA, preferably genomic DNA. The nucleic acid may be indicated herein as a mutant nucleic acid.

Optionally, the nucleic acid of the invention comprises or consists of a GAS-short gene, wherein the sequence of SEQ ID NO: 22, or an analogous sequence thereof, is replaced by any one of SEQ ID NO: 25-36. Optionally, the nucleic acid of the invention comprises or consists of a GAS-short gene, wherein the sequence of SEQ ID NO: 23, or an analogous sequence thereof, is replaced by any one of SEQ ID NO: 37-41 or SEQ ID NO: 71. Optionally, the nucleic acid of the invention comprises or consists of a GAS-long gene, wherein the sequence of SEQ ID NO: 24, or an analogous sequence thereof, is replaced by any one of SEQ ID NO: 42-44.

In a further aspect, the invention encompasses the modified GAS protein as defined in the first aspect, i.e. which is less functional or dysfunctional as compared to the GAS protein encoded by an unmodified GAS gene. The modified GAS protein results in at least one of decreased STL levels, increased squalene levels and increased phenolic compound levels, preferably a combination thereof, preferably a combination of all three, when expressed in a plant, preferably in the absence of expression of a functional GAS protein. In other words, the invention encompasses a GAS protein having a modification that results in a decreased or abolished function, which is capable of at least one of reducing STL levels, increasing squalene levels and increasing phenolic compound levels, preferably thereof, preferably a combination of all three, when expressed in a plant. Preferably the modified GAS protein is a modified endogenous protein of said plant, which is encoded by a modified endogenous GAS gene. Preferably, STL levels of a plant comprising the modified endogenous GAS gene encoding the modified GAS protein of the invention are reduced as compared to the STL levels of a control plant not comprising said modification, when grown under similar conditions. Preferably, squalene levels of a plant comprising the modified endogenous GAS gene encoding the modified GAS protein of the invention are increased as compared to the squalene levels of a control plant not comprising said modification, when grown under similar conditions. Preferably, phenolic compound levels of a plant comprising the modified endogenous GAS gene encoding the modified GAS protein of the invention are reduced as compared to the phenolic compound levels of a control plant not comprising said modification, when grown under similar conditions. Preferably, STL levels are reduced, squalene levels are increased and phenolic compound levels are increased in a plant comprising the modified endogenous GAS gene encoding the modified GAS protein of the invention as compared to respectively the STL levels, the squalene levels and the phenolic compound levels of a control plant not comprising said modification, when grown under similar conditions. Optionally, a plant comprising two or more modified endogenous GAS genes, preferably GAS-short genes, encoding two or more modified GAS proteins, preferably GAS-short proteins, of the invention shows at least one of at least one of reduced STL levels, increased squalene levels and increased phenolic compound levels, preferably a combination thereof, preferably a combination of all three, as compared to a control plant comprising the unmodified endogenous counterparts encoding functional GAS proteins. The activity may be reduced by one or more amino acid insertions, deletions or substitutions. Alternatively, the activity is reduced because of truncation of the protein for instance because of an early stop and/or frame shift in the encoded gene.

The protein of the invention may be produced synthetically, or in vivo (in cell or in planta) for instance by transcription and translation of a construct, optionally comprising a transgene encoding such protein, e.g. a wild type gene modified to encode said protein, or by transcription and translation of an endogenous sequence modified to encoded such protein. Preferably, the protein of the invention is derived from a wild type and/or endogenous GAS protein. The expression of the protein of the invention may be controlled by an endogenous promoter, such as, but not limited to, the promoter naturally controlling the expression of the wild type or endogenous protein from which the protein of the invention is derived.

Preferably, the nucleic acid and/or protein of the invention is present in a plant defined herein. Preferably, the nucleic acid and/or protein of the invention are derived from an endogenous gene and/or protein of said plant.

The invention also relates to a nucleic acid encoding the modified GAS protein of the invention as defined herein.

In a further aspect, the invention provides for a host cell comprising one or more nucleic acids and/or proteins of the invention. Preferably, said host cell comprises one or more, or all, modified GAS-short genes, resulting in a decreased or abolished expression of functional GAS proteins encoded by said GAS-short genes. Preferably said one or more modified GAS-short genes are located within the same locus, i.e. on a single chromosome or homologues chromosome within the host cell.

Preferably, said host cell comprises a modified CiGAS-52 gene, or homologue thereof, wherein preferably both alleles of said gene are modified, resulting in a decreased or abolished expression of functional GAS proteins encoded by said CiGAS-52 gene, or homologue thereof. Optionally, the modification may be in exon 4 of the GAS-S2 gene.

Preferably, said host cell comprises a modified CiGAS-S1 gene, or homologue thereof, wherein preferably both alleles of said gene are modified, resulting in a decreased or abolished expression of functional GAS proteins encoded by said CiGAS-S1 gene, or homologue thereof. Optionally, the modification may be in exon 4 of the GAS-S1 gene.

Preferably, said host cell comprises a modified CiGAS-53 gene, or homologue thereof, wherein preferably both alleles of said gene are modified, resulting in a decreased or abolished expression of functional GAS proteins encoded by said CiGAS-53 gene, or homologue thereof. Optionally, the modification may be in exon 4 of the GAS-S3 gene.

Preferably, said host cell comprises a modified CiGAS-S1 and a modified CiGAS-52 genes, or homologues thereof, wherein preferably both alleles of said genes are modified, resulting in a decreased or abolished expression of functional GAS proteins encoded by said CiGAS-S1 gene and CiGAS-52 gene, or homologues thereof.

Preferably, said host cell comprises a modified CiGAS-S1 gene, a modified CiGAS-52 gene and a modified CiGAS-53 gene, or homologues thereof, wherein preferably both alleles of said genes are modified, resulting in a decreased or abolished expression of functional GAS proteins encoded by said CiGAS-S1 gene, CiGAS-52 gene and CiGAS-53 gene, or homologues thereof. Optionally, said host cell comprises a modified CiGAS-S1 gene, modified CiGAS-52 gene, modified CiGAS-53 gene and modified CiGAS-L1 genes or homologues thereof, wherein preferably both alleles of said genes are modified, resulting in a decreased or abolished expression of functional GAS proteins encoded by said CiGAS-S1 gene, CiGAS-52 gene, CiGAS-53 gene and CiGAS-L1 gene, or homologues thereof. Optionally, the modifications may be in exon 4 of the GAS genes as detailed herein above.

Preferably, said host cell is a plant cell. Even more preferably, said host cell is a plant cell that is desired to have at least one of a reduced STL level, an increased squalene level and increased phenolic compound level, preferably a combination thereof, preferably a combination of all three. Preferably, said host cell is a plant cell that is desired to have at least one of an abolished STL level, an induced squalene level and induced phenolic compound level, preferably a combination thereof, preferably a combination of all three. Said plant cell may be from any plant species. Non-limiting examples of suitable plant species are species belonging to the Asteraceae family, such as of the subfamily Cichorioideae, optionally of the genus of Lactuca (e.g. Lactuca sativa), the genus of Taraxacum (e.g. Taraxacum officinale), the genus of Cichorium (e.g. Cichorium intybus, Cichorium endivia), the genus Scorzonera (e.g. Scorzonera hispanica or Scorzonera humilis), the genus Cynara (e.g. Cynara scolymus), the genus Tragopogon (e.g. Tragopogon porrifolius) or the genus of Gazania. Other suitable examples of species belonging to the Asteraceae family are of the subfamily Asteroideae, such as of the genus Heliantheae (e.g. Helianthus annuus or Helianthus tuberosus), the genus Parthenium (e.g. Parthenium argentatum) or the genus Artemisia (e.g. Artemisia annua). Further suitable plant species as plant species of the Lamiaceae family, Vitaceae family and Cannabaceae family (e.g. see Nguyen et al. Biochem Biophys Res Commun. 2016 Oct. 28; 479(4):622-627 and Want et al. Plant physiology, 2008, 148: 1254-1266).

In an embodiment, the host cell of the invention is produced by at least one of mutagenesis and transformation of a nucleic acid as defined herein. In an embodiment, the host cell can be a mutagenized or transgenic host cell.

In a further aspect, the invention encompasses a method for producing a plant having at least one of reduced STL levels, increased squalene levels and increased phenolic compound levels, preferably a combination thereof, preferably a combination of all three, wherein said method comprises the step of impairing expression and/or activity of one or more functional GAS proteins. Preferably, the method comprises a step of impairing expression and/or activity the GAS-short protein encoded by the CiGAS-52 gene, or homologue thereof. Preferably, the method comprises a step of impairing expression and/or activity the GAS-short protein encoded by the CiGAS-S1 gene, or homologue thereof. Preferably, the method comprises a step of impairing expression and/or activity the GAS-short protein encoded by the CiGAS-53 gene, or homologue thereof.

Preferably, the method comprises impairing expression and/or activity the GAS-short proteins encoded by the CiGAS-S1 and CiGAS-52 gene, or homologues thereof. Preferably, the method comprises impairing expression of the GAS-short proteins encoded by the CiGAS-S1, CiGAS-52 and CiGAS-53, or homologues thereof. Optionally, the method comprises impairing expression of the GAS proteins encoded by the CiGAS-S1, CiGAS-52, CiGAS-53 and CiGAS-L1, or homologues thereof. Impaired expression of functional GAS proteins may comprise genetic modification of endogenous GAS genes as detailed herein above. Optionally expression of functional GAS proteins is reduced by mutating the endogenous GAS gene or genes. Mutating the endogenous GAS gene may result in the expression a dis- or non-functional protein.

Optionally expression of functional GAS proteins is abolished by knocking out the endogenous GAS gene or genes. Knocking out an endogenous GAS gene can be achieved e.g. by T-DNA insertion or introduction of an early stop in the coding sequence.

In case the step of impairing expression of the functional GAS protein in a plant cell or plant tissue, the method may further comprise the step of regenerating the plant cell or plant tissue into a plant. Preferably, said regeneration is performed under conditions that allow for regeneration, preferably said conditions are optimal conditions that allow for regeneration. The method for producing a plant having at least one of reduced STL levels, increased squalene levels and increased phenolic compound levels, preferably a combination thereof, preferably a combination of all three, can also be regarded as at least one of a method for reducing STL levels, increasing squalene levels and increasing phenolic compound levels, preferably a combination thereof, preferably a combination of all three, in said plant. Optionally, the method further comprises at least one of a step of inulin extraction, a step of squalene extraction and a step of phenolic compound extraction, preferably a combination thereof, preferably a combination of all three. The method of the invention may therefore also be regarded as at least one of a method for inulin extraction, squalene extraction and phenolic compound extraction, preferably a combination thereof, preferably a combination of all three, from a plant having reduced STL levels, increased squalene levels and/or increased phenolic compound levels. Alternatively or in addition, the method further comprises harvesting and/or processing the leaves, e.g. for consumption. The method of the invention may therefore also be regarded as a method for producing plant parts, preferably roots or leaves, having reduced STL levels and/or having a reduced bitter taste. The method of the invention may therefore also be regarded as a method for producing plant parts, preferably roots or leaves, having increased phenolic compound levels and/or having increased antioxidant levels.

The invention relates to plant parts, preferably roots or leaves, optionally further processed, for use as a medicament. The invention also relates to plant parts, preferably roots or leaves, optionally further processed, for use in the prevention, amelioration, or treatment of a disease related to oxidative stress, such as, but not limited to heart disease, cancer, arthritis, stroke, respiratory diseases, immune deficiency, emphysema, Parkinson's disease, and/or inflammatory or ischemic conditions.

Alternatively or in addition, introducing expression of the protein of the invention may be achieved by mutating an endogenous GAS gene in a plant, resulting in decreased expression of a functional GAS protein. The GAS endogenous coding sequence may be modified by mutagenesis to result in a sequence encoding the modified GAS protein of the invention. Optionally, the modification results in a non-naturally GAS gene, i.e. a GAS gene that does not occur in nature, and optionally the modification results in expression of a non-natural GAS protein, i.e. a GAS protein not occurring in nature.

The expression of the protein of the invention may be controlled by an endogenous promoter, such as, but not limited to the promoter controlling the expression of an endogenous GAS protein in a control plant. Alternatively or in addition, expression of the protein of the invention may be controlled by a promoter that is not an endogenous promoter, i.e. the promoter sequence is introduced in the plant. Optionally, the method of the invention comprises a step of modifying a regulatory sequence of the gene, such as the promoter sequence resulting in reduced expression of the encoded GAS protein. In such case, expression of a modified or endogenous GAS protein may be controlled by a modified endogenous promoter, wherein said modification results in reduced expression as compared to expression of said protein that is under the control of an unmodified endogenous promoter.

The invention further pertains to a method for at least one of reducing STL levels, increasing squalene levels and increasing phenolic compound levels, preferably a combination thereof, preferably a combination of all three, in a plant as compared to a control plant, comprising treating the plant with one or more compounds that inhibit the activity of the GAS protein, preferably wild-type and/or endogenous GAS protein as defined herein in said plant, preferably inhibiting the activity of at least one or more GAS-short proteins.

The plant of the invention may be a monocot or dicot. Preferably, the plant is of a species belonging to the Asteraceae family, such as of the subfamily Cichorioideae, optionally to the genus of Lactuca (e.g. Lactuca sativa), the genus of Taraxacum (e.g. Taraxacum officinale), the genus of Cichorium (e.g. Cichorium intybus, Cichorium endivia), the genus Scorzonera (e.g. Scorzonera hispanica or Scorzonera humilis), the genus Cynara (e.g. Cynara scolymus), the genus Tragopogon (e.g. Tragopogon porrifolius) or the genus of Gazania. Optionally, the plant is a of the subfamily Asteroideae, such as of the genus Heliantheae (e.g. Helianthus annuus or Helianthus tuberosus), the genus Parthenium (e.g. Parthenium argentatum), or the genus Artemisia (e.g. Artemisia annua). The plant may also be a plant of the Lamiaceae family, Vitaceae family and Cannabaceae family (e.g. see Nguyen et Biochem Biophys Res Commun. 2016 Oct. 28; 479(4):622-627).

The plant may be, or may be obtainable from, the Asteraceae family, preferably of the subfamily of Cichorioideae, preferably of the genus Cichorium, more preferably an Cichorium intybus plant, and preferably the one or more modified GAS genes of the method of the invention comprises at least one modified GAS-short gene derived from a gene that is, or is a homologue of, a gene comprising a coding sequence of any one of SEQ ID NO: 7-12, and/or preferably the one or more modified GAS proteins of the method of the invention comprises at least one GAS-short protein that is derived from a protein that is, or is a homologue of, a protein having an amino acid sequence of any one of SEQ ID NO: 1-6. Alternatively or in addition, the one or more modified GAS genes of a method of the invention comprises a GAS-long gene that is derived from a gene that is, or is a homologue of, a gene comprising a coding sequence of SEQ ID NO: 14 and/or the one or more modified GAS proteins of the method of the invention comprises a modified GAS-long protein that is derived from a protein that is, or is a homologue of, a protein having an amino acid sequence of SEQ ID NO: 13.

Optionally, the method of the invention further comprises a step for transferring the one or more modified GAS genes of the invention (the one or more nucleic acids of the invention) to offspring of the plant produced by the method of the invention, which may be performed by introgression. Breeding techniques for introgression are well known to one skilled in the art.

Preferably, the method of the invention results in a plant having at least one of reduced STL levels, increased squalene levels and increased phenolic compound levels, preferably a combination thereof, preferably a combination of all three, compared to a control plant as defined herein.

The method of the invention may further comprise a step of screening or testing the plant for reduced or abolished levels of functional GAS protein and/or for at least one of reduced STL levels, increased squalene levels and increased phenolic compound levels. Preferably, the method of the invention may further comprise a step of screening or testing the plant for reduced or abolished levels of functional GAS protein together with a combination, or all three, of reduced STL levels, increased squalene levels and increased phenolic compound levels. Any screening or testing method known in the art can be used for screening the plant, such as, but not limited to, the methods described herein. Said screening or testing can be assessing expression of functional and/or modified GAS protein at a molecular level (protein or mRNA) or assess the presence of a nucleic acid or construct comprising the modified GAS gene of the invention and/or encoding the modified GAS protein of the invention. The person skilled in the art is aware of techniques to assess protein expression and/or the presence or absence of a nucleic acid sequence within a plant.

The method for producing a plant of the invention having at least one of reduced STL levels, increased squalene levels and increased phenolic compound levels, preferably a combination thereof, preferably a combination of all three, as defined herein may further comprise a step of assessing expression or the protein of the invention and/or detecting the presence of the nucleic acid of the invention in said plant and optionally subsequently selecting said plant.

Expression of the protein of the invention can be determined using any conventional method known to the skilled person. Such methods include detecting the transcript (e.g. mRNA) or detecting the protein of the invention or detection of the enzyme activity for instance by detecting products of the reaction catalyzed by the enzyme. Non-limiting examples for detecting the transcript include e.g. PCR, q-PCR and northern blotting. Non-limiting examples for detecting the presence of the protein of the invention includes e.g. western blotting and mass spectrometry on full polypeptides and peptide digests. The person skilled in the art is also aware of using methods for screening for the presence of the nucleic acid of the invention. The person in the art is well aware of molecular techniques to identify such sequences, e.g. Sequence Based Genotyping (Hoa T. Truong, A. Marcos Ramos, Feyruz Yalcin, Marjo de Ruiter, Hein J. A. van der Poel, Koen H. J. Huvenaars, Rene C. J. Hogers, Leonora. J. G. van Enckevort, Antoine Janssen, Nathalie J. van Orsouw, and Michiel J. T. van Eijk. Sequence-Based Genotyping for Marker Discovery and Co-Dominant Scoring in Germplasm and Populations. PLoS One. 2012; 7(5): e37565), oligo-ligation (SNPSelect; Rene C. J. Hogers, Marjo de Ruiter, Koen H. J. Huvenaars, Hein van der Poel, Antoine Janssen, Michiel J. T. van Eijk, Nathalie J. van Orsouw. SNPSelect: A scalable and flexible targeted sequence-based genotyping solution; PLoS One. 2018; 13(10): e0205577), AFLP (Zabeau, M. and Vos, P. (1993) Selective restriction fragment amplification; a general method for DNA fingerprinting; Vos, P., Hogers, R., Bleeker, M., Reijans, M., van de Lee, T., Hornes, M., Frijters, A., Pot, J., Peleman, J., Kuiper, M. et al. (1995) AFLP: a new technique for DNA fingerprinting. Nucl. Acids Res., 21, 4407-4414), and the like.

As also indicated herein above, the method may further comprise a step of producing progeny of the plant comprising the nucleic acid of the invention and/or expressing the protein of the invention. The method can comprise a further step of producing seeds from the plant expressing the protein of the invention. The method may further comprise growing the seeds into plants that comprise the nucleic acid and/or protein of the invention.

In a further aspect, the invention relates to a method of screening plants comprising one or more nucleic acids of the invention and/or expressing one or more proteins of the invention. Said method comprises a step of assessing the presence of the nucleic acid of the invention in said plant and/or assessing expression of the protein of the invention in said plant and optionally subsequently selecting said plant cell, plant tissue or plant, preferably as described herein above.

In a further aspect, provided it is a plant comprising one or more proteins, nucleic acids and/or constructs of the invention, and a plant obtainable from a method as defined herein. The plant may comprise a modification resulting in impaired expression of a functional GAS protein, wherein the modification is in one or more, preferably all endogenous genomic GAS-short genes, optionally all endogenous genomic GAS genes. Preferably said one or more GAS-short genes are located within the same locus, i.e. on a single chromosome or homologues chromosome. The plant may comprise a mutation in one or more, optionally all, endogenous genomic GAS genes, wherein the mutation results in the impaired expression of a functional GAS protein. The plant may comprise a mutation in one or more, optionally all, endogenous functional GAS-short genes, wherein the mutation results in the impaired expression of a functional GAS protein. Preferably, it comprises such modification in at least the CiGAS-52 gene, or a homologue thereof, preferably both alleles of said gene. Preferably, it comprises such modification in at least the CiGAS-S1 gene, or a homologue thereof, preferably both alleles of said gene. Preferably, it comprises such modification in at least the CiGAS-53 gene, or a homologue thereof, preferably both alleles of said gene. Preferably, it comprises such modification in the in both the CiGAS-S1 and the CiGAS-52 genes, or homologues thereof, preferably both alleles of these genes. Preferably, it comprises such modification in the CiGAS-S1, CiGAS-52 and CiGAS-53 genes, or homologues thereof, preferably in both alleles of these genes. Optionally, it comprises such modification in the CiGAS-S1, CiGAS-S2, CiGAS-S3 genes and CiGAS-L1 genes, or homologues thereof, preferably in both alleles of these genes. Thus the plant cell, plant tissue and/or plant of the invention may be characterized by one or more, optionally all, modified GAS-short proteins, optionally one or more disrupted GAS-short proteins, which shows a decreased or lost function and/or activity. Optionally, the plant cell, plant tissue and/or plant of the invention further comprises one or more modified GAS-long proteins, optionally one or more disrupted GAS-long proteins, which shows a decreased or lost function and/or activity.

Further, the plant cell, plant tissue and/or plant of the invention may be characterized by a reduced or abolished expression of an endogenous GAS protein, preferably a GAS-short protein. The plant comprising the one or more modified GAS genes of the invention and/or the one or more modified GAS proteins of the invention has at least one of reduced STL levels, increased squalene levels and increased phenolic compound levels, preferably a combination thereof, preferably a combination of all three, as compared to a control plant cell, plant tissue or plant, which can be tested for and/or screened for as indicated herein. Optionally the plant cell, tissue or plant of the invention is a root.

As a non-limiting example, the reduced STL levels can be determined by comparing a control plant with a plant of the invention, under controlled conditions chosen such that in the control plant a significant level of one or more STLs can be observed, preferably an STL as defined herein. In a further example, the increased squalene levels can be determined by comparing a control plant with a plant of the invention, under controlled conditions chosen such that the control plant has a low or undetectable level of squalene. In another example, the increased phenolic compound levels can be determined by comparing a control plant with a plant of the invention, under controlled conditions chosen such that the control plant has a limited level of a phenolic compound.

The combination of reduced STL levels, increased squalene levels and increased phenolic compound levels can be determined by comparing a control plant with a plant of the invention, under controlled conditions chosen such that in the control plant a significant level of one or more STLs can be observed and the control plant has a low or undetectable level of squalene and limited levels of phenolic compounds.

When a plant has at least one of a reduced STL levels, increased squalene levels and increased phenolic compound levels, or a combination thereof, or a combination of all three, it is preferably capable of sustaining a normal growth and/or a normal development. At least one of reduced STL levels, increased squalene levels and increased phenolic compound levels, or a combination thereof, or a combination of all three, can be determined by comparing plants. As a non-limiting example, one plant of the invention may be compared with one control plant. Alternatively or in addition, a group of plants of the invention may be compared with a group of control plants. Each group can comprise e.g. at least about 2, 3, 4, 5, 10, 15, 20, 25, 50 or 100 individual plants.

The skilled person is well aware how to select appropriate conditions to determine at least one of STL levels, squalene levels and phenolic compound levels, or a combination thereof, or a combination of all three, and how to measure at least one of a reduction of STL levels, an increase of squalene levels and an increase of phenolic compound levels, or a combination thereof, or a combination of all three.

The plant may be a transformant and/or mutant, i.e. not being a wild type or naturally occurring plant cell tissue or plant as it comprises a modified GAS gene and/or expresses a modified GAS protein.

In an embodiment, the plant and/or host cell of the invention is not, or is not exclusively, obtained by an essentially biological process.

Preferably, the plant of the invention and/or of the method of the invention may be a crop plant or a cultivated plant, i.e. plant species which is cultivated and bred by humans. A crop plant may be cultivated for food or feed purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork, fibres (such as cotton) and the like. Preferably, the plant part, plant cell, seed, and/or rootstock as taught herein are from a crop plant.

The plant cell, tissue or plant may be, or may be obtainable from, a plant of a species belonging to the Asteraceae family, Lamiaceae family, Vitaceae family and Cannabaceae family, preferably of the Asteraceae family, such as of the subfamily Cichorioideae, optionally to the genus of Lactuca (e.g. Lactuca sativa), the genus of Taraxacum (e.g. Taraxacum officinale), the genus of Cichorium (e.g. Cichorium intybus, Cichorium endivia), the genus Scorzonera (e.g. Scorzonera hispanica or Scorzonera humilis), the genus Cynara (e.g. Cynara scolymus), the genus Tragopogon (e.g. Tragopogon porrifolius) or the genus of Gazania, or optionally of the subfamily Asteroideae, such as of the genus Heliantheae (e.g. Helianthus annuus or Helianthus tuberosus), the genus Parthenium (e.g. Parthenium argentatum), or the genus Artemisia (e.g. Artemisia annua), and preferably the modified GAS gene of the invention is derived from a gene that is, or is a homologue of, a gene comprising a coding sequence of any one of SEQ ID NO: 7-12 and 14, preferably of any one of SEQ ID NO: 7-12, and/or the modified GAS protein of the invention is derived from a protein that is, or is a homologue of, a protein having an amino acid sequence of any one of SEQ ID NO: 1-6 and 13, preferably any one of SEQ ID NO: 1-6.

A further aspect of the invention pertains to seeds produced by the plant having at least one of reduced STL levels, increased squalene levels and increased phenolic compound levels, or a combination thereof, or a combination of all three, as defined herein and comprising one or more modified GAS genes and/or one or more modified GAS proteins of the invention.

An additional aspect of the invention pertains to plants grown from the seeds or regenerated from the plant cell, comprising one or more nucleic acids and/or one or more proteins of the invention as defined herein.

An additional aspect of the invention described herein pertains to progeny of the plant of the invention, wherein the progeny has at least one of reduced STL levels, increased squalene levels and increased phenolic compound levels, or a combination thereof, or a combination of all three, as specified herein and wherein the progeny comprises one or more nucleic acids and/or proteins of the invention. The progeny may be obtained by selfing or breeding and selection, wherein the selected progenies retain at least one of the reduced STL biosynthesis, increased squalene accumulation and increased phenolic compound accumulation, or a combination thereof, or a combination of all three, of the parent plant and/or retain nucleic acid and/or protein of the invention.

In an aspect, the invention further concerns the use of a nucleic acid, protein, construct, or host cell of the invention for at least one of reducing STL levels, increasing squalene levels and increasing phenolic compound levels, or a combination thereof, or a combination of all three, in a plant.

In an aspect, the invention pertains to plant parts and plant products derived from the plant of the invention and/or plant obtained or obtainable by the method of the invention, wherein the plant part and/or plant product comprise one or more modified GAS genes, preferably modified GAS-short genes and/or one or more modified GAS proteins, preferably modified GAS-short proteins and/or parts thereof. Such plant parts and/or plant products may be seed or fruit and/or products derived therefrom. Such plant parts, plant products may also be non-propagating material.

The present invention has been described above with reference to a number of exemplary embodiments as shown in the drawings. Modifications and alternative implementations of some parts or elements are possible, and are included in the scope of protection as defined in the appended claims.

### BRIEF DESCRIPTION OF THE FIGURES

FIG. 1: Alignment of exon 4 sequences of Cichorium intybus GAS genes and indication of the sequence targeted by the guide RNAs. The underlines indicate the target sequences of the guide RNAs (the sequence of GAS-S1, GAS-S2, GAS-S3 and GAS-L correspond to respectively SEQ ID NOs: 67, 68, 69 and 70).

FIG. 2: Indel mutations of the five selected mutant lines (MT1 to MT5) are shown. For each gene the target site is shown underlined with the mutations present in each allele shown underneath each target. Alleles without indels are indicated as wild type.

FIG. 3: STL (lactucin, lactucopicrin and 8-deoxylactucin) levels expressed in leaves of the different mutant (MD and control lines (WT). The genotypes for each line is provided in the table underneath the x-axis, wherein a “+” means a wild type allele, and a “−” means a mutated allele. Specific mutations are provided in FIG. 2.

FIG. 4: STL (lactucin 15-oxalate, lactucopicrin 15-oxalate and 8-deoxylactucin 15-oxalate) levels in leaves of the different mutant control lines. The genotypes for each line is provided in the table underneath the x-axis, wherein “+” means a wild type allele, and “−” means a mutated allele. Specific mutations are provided in FIG. 2.

FIG. 5: STL (lactucin, lactucopicrin and 8-deoxylactucin) levels in roots of the different mutant (MT) and control lines NOT The genotypes for each line is provided in the table underneath the x-axis, wherein a “+” means a wild type allele, and a “−” means a mutated allele. Specific mutations are provided in FIG. 2.

FIG. 6: STL (lactucin 15-oxalate, lactucopicrin 15-oxalate and 8-deoxylactucin 15-oxalate) levels in roots of the different mutant control lines. The genotypes for each line is provided in the table underneath the x-axis, wherein “+” means a wild type allele, and “−” means a mutated allele. Specific mutations are provided in FIG. 2.

FIG. 7: GC-MS chromatogram of chicory root tissues. Peak1-3: acetylated triterpenes; Peak 4: squalene; Peak 5: stigmasterol; Peak 6: sitosterol.

FIG. 8: Increase of phenolic compounds in leaves and roots of chicory GAS KO lines.

### EXAMPLE 1

In this example we describe the isolation of protoplasts from chicory leaves which were then transfected with different CRISPR/Cas9 reagents targeting the GAS genes, in order to also investigate efficiency of these different reagents and feasibility of the technology to induce indels in multiple genes and alleles at the same time in chicory. These protoplasts were then regenerated into mature plants with mutations in different GAS genes which were then phenotyped for STL content. Surprisingly, we found that inactivation of the GAS-short genes without affecting the GAS-long gene, almost fully blocked STL production in both leaf and root tissue.

### Isolation of Chicory Protoplasts

Protoplast isolation, transfection and culture was performed as previously described (Frearson et al. Dev Biol, 1973, 33, 130-137; Kao et al. Planta, 1975, 126, 105-110; Negrutiu et al. Plant. Mol. Biol. 1978, 8, 363-373; Nenz et al. Plant Cell Tiss Org, 2000, 62, 85-88; Deryckere et al. Plant Cell Rep 2012, 31, 2261-2269) with several modifications. In vitro shoot cultures of Cichorium intybus var. sativa (Orchies C37) were maintained on MS20 medium with 0.8% agar in high plastic jars at 16/8 h photoperiod of 100 μmol·m⁻²·s⁻¹ PPF at 25° C. and 60-70% RH. Young leaves (10-12) were harvested, placed in a dish containing 5 ml CPW9M medium (Frearson et al. Dev Biol, 1973, 33, 130-137) and were gently sliced perpendicularly to the mid nerve to ease the penetration of the enzyme mixture. Sliced leaves were transferred to a dish containing 25 ml CPW9M and an enzyme mixture (1% (w/v) Cellulase Onozuka RS, 0.2% (w/v) Macerozyme Onozuka R10). Digestion was carried out at 25° C. for 14-16 h, in the dark. The protoplasts were filtered through a 50 μm stainless steel sieve and were harvested by centrifugation for 5 minutes at 85×g. Protoplasts were resuspended in 1 ml CPW9M medium and then added to a tube containing 5 ml CPW13S-1.2M. This was then centrifuged for 10 minutes at 85×g at RT. Live protoplasts were then harvested from the interface layer, transferred to a fresh tube and then mixed with 11 ml CPW9M. The protoplast density was then determined in a haemocytometer.

### Transfection and CRISPR/Cas9 Mutagenesis of Chicory GAS Genes

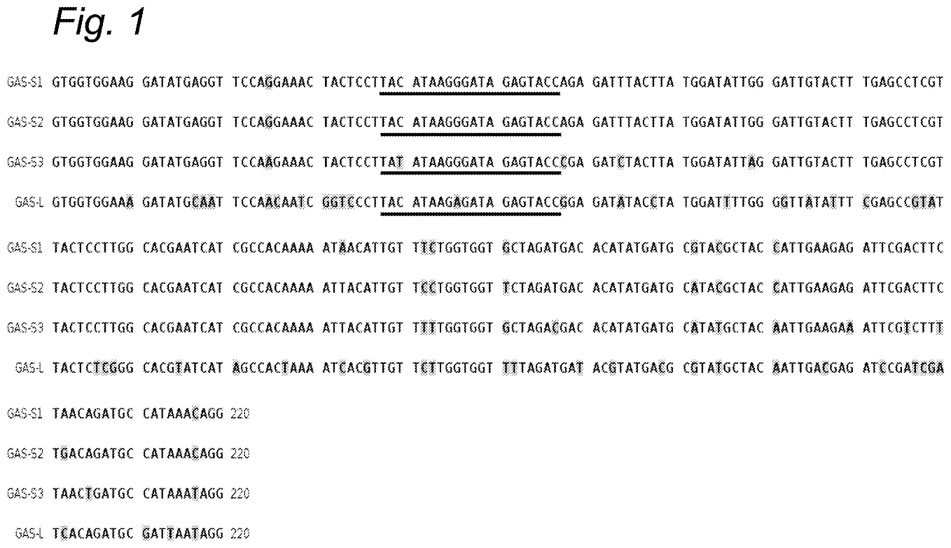

Exon 4 of the GAS-family enzymes encodes a region of the protein that makes up part of the GAS active site. Chicory leaf protoplasts were transfected either with CRISPR-Cas9/guide RNA complexes (RNPs) or plasmids encoding the same using guide RNAs targeting exon 4 in the GAS-short and GAS-long genes (i.e. targeting SEQ ID NO: 22, 23 and 24, respectively; see also FIG. 1). RNPs were made by combining 10 μg SpCas9-NLS protein (New England Biolabs) and 10 μg of a guide RNA in 1×SpCas9 reaction buffer (New England Biolabs) in a final volume of 20 μl. For plasmid based transfection, a plasmid encoding the guide RNAs operably linked to an Arabidopsis U6 promoter and a plasmid carrying the SpCas9 ORF operably linked to an Arabidopsis ubiquitin promoter promoter were mixed at a 1:3 molar ration. For each transfection the reagents, i.e. 20 μg RNPs or 80 μg plasmids encoding the same, were mixed with 0.25×10⁶ protoplasts in a total volume of 250 μl MaMg medium and 250 μL PEG solution (400 g/l poly(ethylene glycol) 4000, Sigma-Aldrich #81240; 0.1 M Ca(NO₃)₂) was then added. The transfection was then allowed to take place for 20 minutes at room temperature followed by the addition of 5 ml 0.275 M Ca(NO₃)₂ solution which was thoroughly, but gently mixed in. The protoplasts were harvested by centrifugation for 5 minutes at 85×g and resuspended in 0.25 ml 9M culture medium.

### Generation of GAS Mutant Plants

Transfected protoplasts were centrifuged at 85×g for 5 minutes at RT and then resuspended at a density of 0.10×10⁵ cells/ml in 5 ml 9M medium. An equal volume of alginate solution was then added dropwise and mixed thoroughly, and 1 ml of the mixture was then layered on a Ca-Agar plate (5 cm dish), dispersing the mixture evenly over the whole plate surface to form a disc. The alginate was allowed to polymerize for one hour and was then transferred to a 5 ml culture dish containing 4 ml K1Cg medium. After 7 days of culture in the dark at 28° C. the liquid culture medium was replaced with 4 ml K5CgK medium and the discs were cultured for a further 7 days using the same conditions. The discs were then cut into 5 mm broad strips and transferred to 9 cm plates with B5g-10-0, 2-SP-NB medium, two discs per plate. These were then incubated at 25° C. in the dark for two to three weeks whereupon the microcalli formed were then picked with tweezers and transferred to MS10-IB plates and incubated at 25° C. under low light for the first week followed by full light for the reminder of the regeneration. Calli were transferred to fresh MS10-IB medium every 3-4 weeks until signs of regeneration appeared. The developing shootlets were harvested and rooted on MS20 medium. Regenerated plants were then genotyped for mutations in the different GAS genes.

### Genotyping Chicory Plants

Genomic DNA was isolated from regenerated chicory plants using the Maxwell Plant DNA kit (Promega) and the target sites in each gene were then amplified separately using specific forward primers (SEQ ID NO: 45 for GAS-S1, SEQ ID NO: 46 for GAS-S2, SEQ ID NO: 47 for GAS-S3 and SEQ ID NO: 48 for GAS-L1) and reverse primers (SEQ ID NO: 49 for GAS-S1, SEQ ID NO: 50 for GAS-S2, SEQ ID NO: 51 for GAS-S3 and SEQ ID NO: 52 for GAS-L1) primers. A nested PCR was then done on each PCR product using the appropriate forward primers (SEQ ID NO: 53 for GAS-S1 and GAS-S2, SEQ ID NO: 54 for GAS-S3 and SEQ ID NO: 55 for GAS-L1) and reverse primers (SEQ ID NO: 56 for GAS-S1 and GAS-S2, SEQ ID NO: 57 for GAS-S3 and SEQ ID NO: 58 for GAS-L1) and a final third PCR was then done with barcoded Illumina primers to enable later identification of the sequences. All of the these PCR products were then pooled and paired-end sequenced on an Illumina MiSeq apparatus. The sequences were then analyzed for the presence of indel mutations at the target sites.

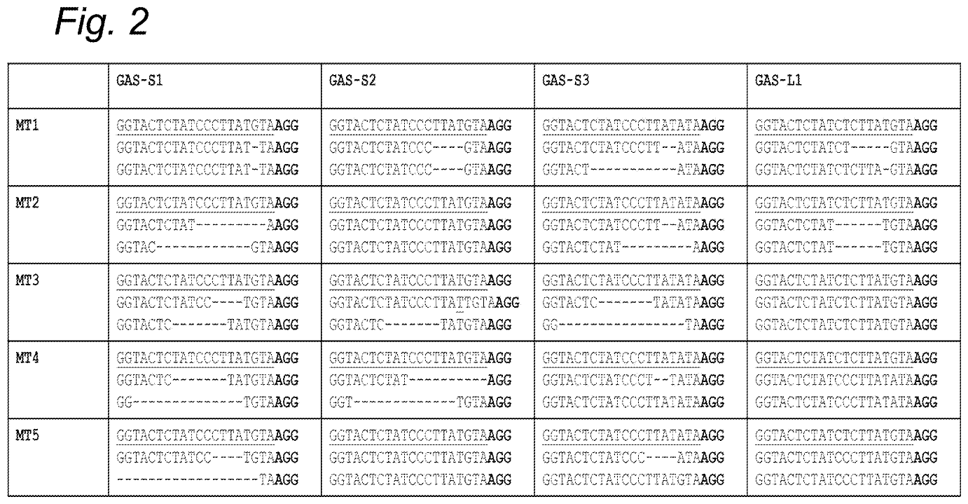

Three mutant lines were selected, MT1, MT2, MT3, MT4 and MT5. As indicated in more detail in FIG. 2, MT1 comprises mutations in all alleles of all four GAS genes; MT2 comprises mutations in all GAS alleles except for the GAS-S2 alleles, which has the wild type sequence; MT3 comprises mutations in all GAS-short alleles, while the two GAS-L1 alleles do not comprise a mutation; MT4 comprises mutations in both GAS-S1 and GAS-S2 alleles and in one GAS-S3 allele, while the GAS-L1 alleles and one GAS-S3 allele did not comprise a mutation; and M5 comprises mutations only in both GAS-S1 alleles and one GAS-S3 allele, while the other GAS alleles do not comprise mutations.

The selected lines were transferred to the greenhouse for further phenotypic analysis. As controls, lines were also selected, which had also been regenerated from protoplasts but lacked any mutations in the GAS genes.

### Quantification of Sesquiterpene Lactone Guaianolides

Sesquiterpene lactone content was determined in the leaves and roots of the five GAS mutant lines and the control plants. Chicory leaf and root material (100 mg) was frozen and powdered in liquid nitrogen. Extraction was performed using 77% methanol containing formic acid (0.1%), the samples were then vortexed, sonicated for 15 min and then centrifuged at 21000 g at room temperature.

The clear supernatant was transferred to a fresh vial and used for LC-MS analysis. LC-MS analysis was performed using the LC-PDA-LTQ-Orbitrap FTMS system (Thermo Scientific) which consist of an Acquity UPLC (H-Class) with Acquity elambda photodiode array detector (220-600 nm) connected to a LTQ/Orbitrap XL hybrid mass spectrometer equipped with an electrospray ionizator (ESI). The injection volume was 5 μl. Chromatographic separation was on a reversed phase column (Luna C18/2, 3μ, 2.0×150 mm; Phenomenex, USA) at 40° C. Degassed eluent A [ultra-pure water: formic acid (1000:1, v/v)] and eluent B [acetonitrile:formic acid (1000:1, v/v)] were used at a flow rate of 0.19 ml min-1. A linear gradient from 5 to 75% acetonitrile (v/v) in 45 min was applied, which was followed by 15 min of washing and equilibration. FTMS full scans (m/z 90.00-1350.00) were recorded with a resolution of 60,000.

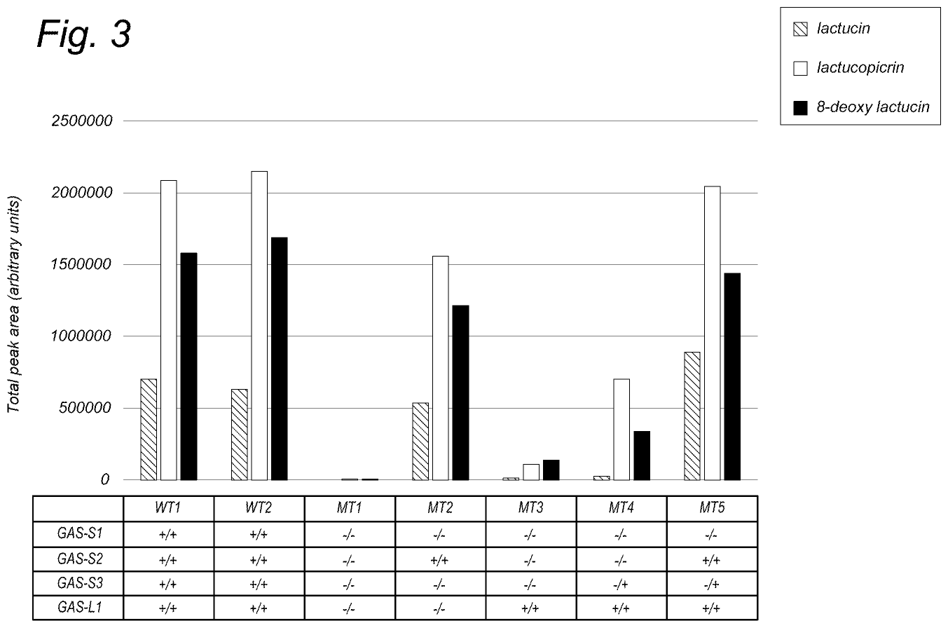

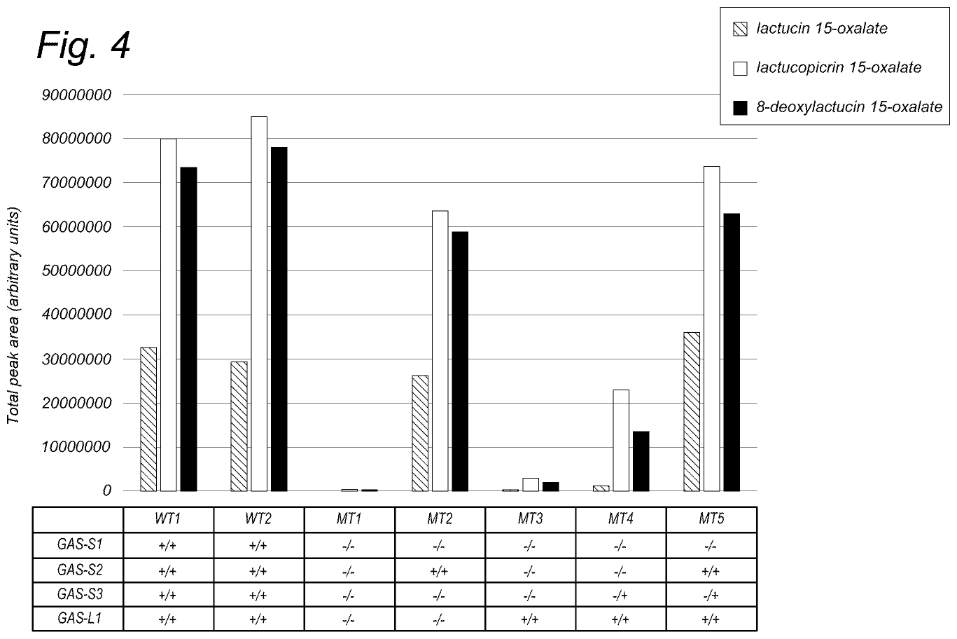

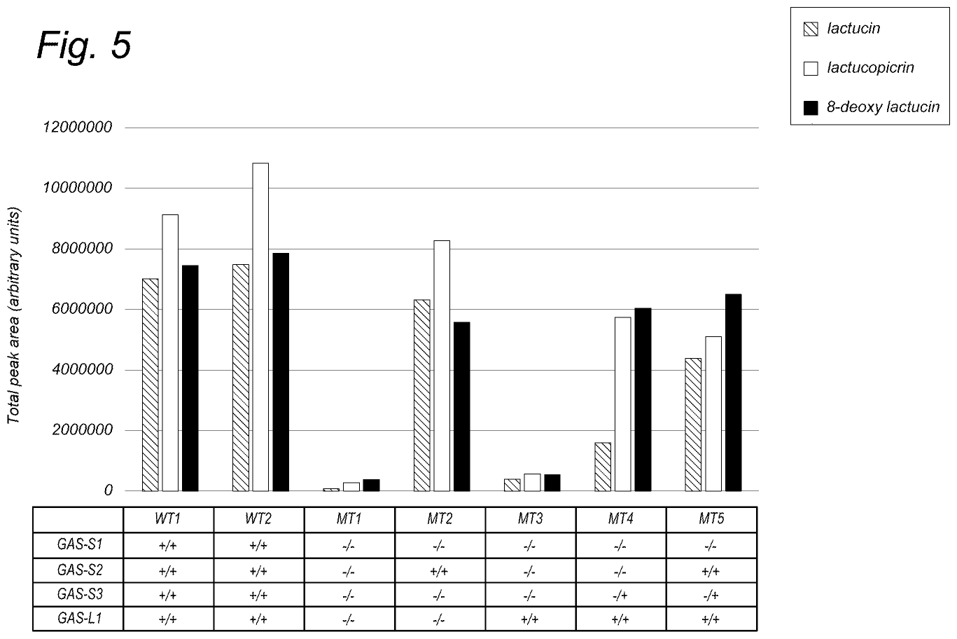

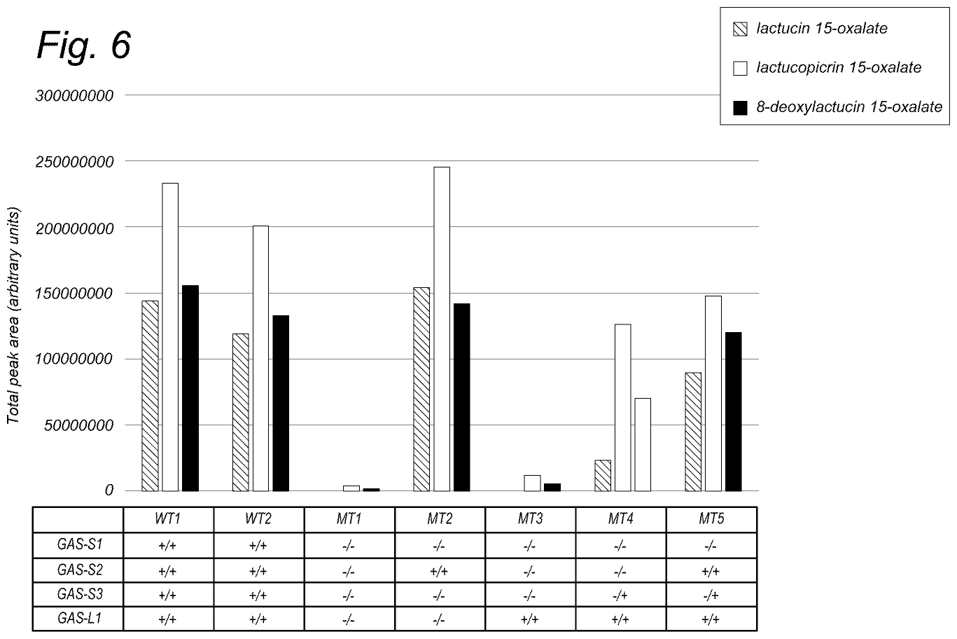

The samples were analyzed for the presence of six STLs (lactucin, lactucin-15-oxalate, 8-deoxylactucin, 8-deoxylactucin 15-oxalate, lactucopicrin and lactucopicrin 15-oxalate). The levels of these compounds in the leaves of the mutant and control plants are shown in FIGS. 3 and 4. The levels of these compounds in the root of the mutant and control plants are shown in FIGS. 5 and 6. The total peak area of each compound was quantified.

### Results

The level of STLs in the two control lines was broadly similar, showing that the regeneration process had not introduced a large amount of STL variation. However, several lines containing mutations in the GAS genes showed a strong reduction in the amount of STLs produced in the leaves and roots. There appears to be a direct correlation between the type of functional GAS genes present and the levels of STLs produced. MT1, containing mutations in all of the GAS genes, shows the lowest STL levels, while the next highest expresser (MT3), lacks functional copies of the GAS-S1/S2/S3 genes but retains the GAS-L1 gene. M4, only lacking the GAS-S1/S2 genes and one GAS-S3 allele, shows reduced STL production by approximately 70%. These results demonstrate that the GAS-S1 and GAS-S2 genes seem to be responsible for most of the STL production in the leaves and roots, with the lines lacking both of these genes (MT1, MT3 and MT4) showing the largest decreased STL levels. MT2, having two functional GAS-S2 alleles still produces approximately 75% of the wild type levels, while MT1 that lacks any functional GAS gene, production is almost eliminated, suggesting that GAS-S2 is most important for sesquiterpene lactone production in the leaves and root. The activity of GAS-L1 seems to be low, as shown by the difference between the MT3 only having retained the GAS-L1 gene and MT1 lacking functional copies of all GAS genes.

Surprisingly, these results are in contrast to the state of the art that suggests GAS-long to be the most relevant GAS gene for STL accumulation. This study shows that inactivation of only one or two GAS-short genes significantly reduces the production of all the STLs assayed to approximately the same extent. Inactivation of all the GAS-short genes nearly abolished STL production in both leaf and root tissue. Several studies have shown that the GAS-long gene is predominantly expressed in leaves, but surprisingly this data demonstrates that mutations in the GAS-short genes have a greater effect on STL accumulation in the leaves and root than mutations in the GAS-long gene and therefore should be targeted to decrease STL levels in chicory.

### EXAMPLE 2

### Squalene Accumulation in Chicory GAS KO Lines

Chicory root and leaf material (300 mg) from 2 WT chicory plants (WT1 and WT2; see Example 1) and 5 edited chicory plants (MT1, MT2, MT3, MT4, MT5; see Example 1) carrying a deletion of the GAS synthase gene was analyzed. Plant material was frozen and powdered in liquid N₂. The samples were then extracted with 1.5 ml of hexane:ethyl acetate mixture (v/v 85:15). Samples were sonicated for 15 min in a sonication bath and centrifuged for 10 min at 1200 rpm. The extracts were dried over a Na₂SO₄ column prepared in a glass wool plugged glass pipette. Analytes from 1 μL samples were separated using a gas chromatograph (5890 series II, Hewlett-Packard) equipped with a 30 m×0.25 mm, 0.25 mm film thickness column (ZB-5, Phenomenex) using helium as carrier gas at flow rate of 1 ml/min. The injector was used in splitless mode with the inlet temperature set to 250° C. The initial oven temperature of 45° C. was increased after 1 min to 310° C. at a rate of 10° C./min and held for 5 min at 300° C. The GC was coupled to a mass-selective detector (model 5972A, Hewlett-Packard), scanning from 45 to 500 atomic mass units. Experimental samples were compared with authentic standards of squalene (Sigma-Aldrich), campesterol (Sigma-Aldrich), stigmasterol (Extrasynthese) and sitosterol (Extrasynthese) for verification.

In the semi-polar methanolic extract of chicory roots the effect of GAS deletion on accumulation of sesquiterpene lactones was studied by LC-MS, as described in Example 1.

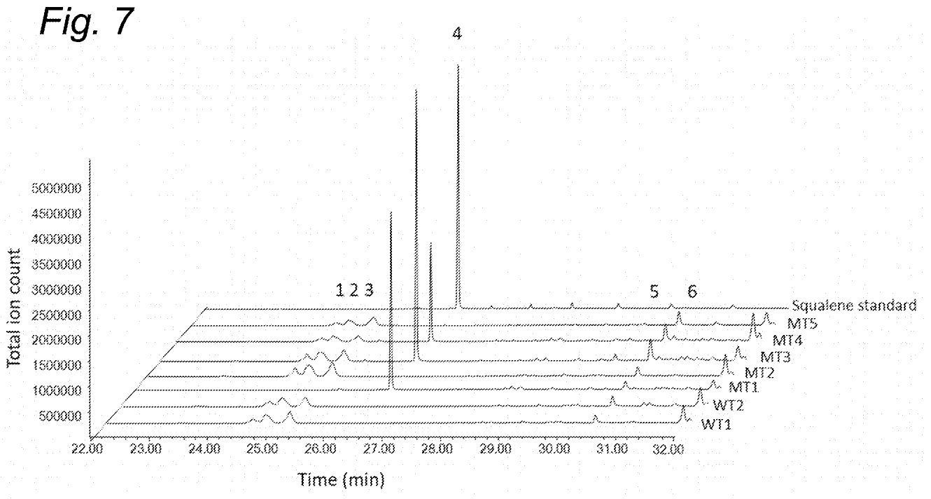

The hexane extract of chicory root was examined for accumulation of terpenes and sterols by GC-MS. Other than trace amounts of farnesene and farnesol in the chicory lines having the highest reduction of STLs (MT1, MT3 and MT4), no accumulation of monoterpenes and sesquiterpenes was observed as compared to WT lines. However, a large new peak was detected in the chromatogram of these lines at the retention time of 26.7 min (see FIG. 7). This compound was identified as squalene by comparison of the mass spectrum to the NIST mass spectral library. The identification was verified by comparison of the retention time and mass spectrum to the authentic standard of squalene. The amount of squalene accumulating in the root was quantified at 154 ug/gFW, 99 ug/gFW and 55 ug/gFW in chicory lines MT3, MT1 and MT4, respectively. No squalene peak was observed in chicory root extracts of lines MT2 and MT5 nor in the extract of the wild-type chicory plants. Therefore, it seems that farnesyl pyrophosphate (FPP, C15) in the chicory roots that would normally be converted to germacrene A by activity of GAS enzymes became available and was converted by the activity of endogenous chicory squalene synthase to squalene (C30).

Squalene is a precursor for the biosynthesis of triterpenes and phytosterols. Wild-type chicory roots accumulate small amounts of acetylated-triterpenes (peak 1-3, elemental formula C32H5202, MW=468; see FIG. 7). Upon comparison of the wild-type plants to the GAS KO lines no increase in the amount of triterpenes was observed in any of the KO lines. The accumulation of phytosterols sitosterol, campesterol and stigmasterol in GAS KO lines was next compared to the WT chicory plants. Sitosterol was the major observed sterol in the root tissue of WT chicory plants (see FIG. 7). In lines MT3 and MT4 2.3-fold and 1.7-fold increase in the level of sitosterol was observed compared to the WT lines, yielding 42 ug/g FW and 32 ug/g FW sitosterol, respectively. WT levels of sitosterol were observed for line MT1, MT2 and MT5. The amount of stigmasterol and campestrol was below 5 ug/g FW for both WT and KO lines and therefore close to the detection limit of the GC-MS method and was not quantified (see FIG. 7).

The GC-MS analysis of the chicory leaves revealed that squalene accumulated to a much lesser extend in the leaves of chicory. In line MT1 only a very minor accumulation of squalene was detected at 13 ug/g FW and the other KO lines did not show increased squalene accumulation in the leaves. No additional accumulation of monoterpenes, sesquiterpenes, triterpenes or sterols beyond WT levels was observed in the leaves of chicory GAS KO lines.

### EXAMPLE 3

### Increase of Phenolic Compounds in Chicory GAS KO Lines

Chicory leaf and root material (100 mg) of the WT1, WT2, MT1, MT2, MT3, MT4, MT5 plants (see Example 1) was frozen and powdered in liquid N₂. Extraction was performed using 77% methanol containing formic acid (0.1%), the samples were then vortexed, sonicated for 15 min and centrifuged at 21000 g at room temperature. The clear supernatant was transferred to a fresh vial and used for LC-MS analysis. LC-MS analysis was performed using the LC-PDA-LTQ-Orbitrap FTMS system (Thermo Scientific) which consist of an Acquity UPLC (H-Class) with Acquity elambda photodiode array detector (220-600 nm) connected to a LTQ/Orbitrap XL hybrid mass spectrometer equipped with an electrospray ionizator (ESI). The injection volume was 5 μl. Chromatographic separation was on a reversed phase column (Luna C18/2, 3˜, 2.0×150 mm; Phenomenex, USA) at 40° C. Degassed eluent A [ultra-pure water: formic acid (1000:1, v/v)] and eluent B [acetonitrile:formic acid (1000:1, v/v)] were used at a flow rate of 0.19 ml min-1. A linear gradient from 5 to 75% acetonitrile (v/v) in 45 min was applied, which was followed by 15 min of washing and equilibration. FTMS full scans (m/z 90.00-1350.00) were recorded with a resolution of 60,000.

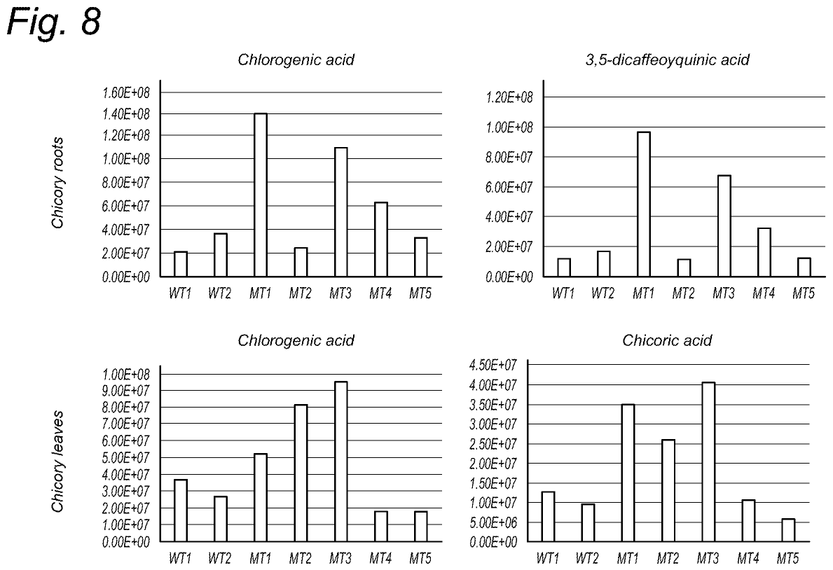

The PDA spectrum of the samples was examined at the wavelength of 320 nm for detection of phenolic compounds. In the chicory root tissues 3,5-dicaffeoylquinic acid (elemental formula C25H24O12, [M+H]⁺=517,13405) and chlorogenic acid (elemental formula C16H1809, [M+H]⁺=355,10235) were observed as major phenolic compounds. In chicory leaves the major accumulated phenolic compounds observed were chlorogenic acid and chicoric acid (Peak 3, C22H18012, [M+H]⁺=475.08710). The compounds were identified by accurate mass determination and comparison with authentic standards of chicoric acid, chlorogenic acid and 3,5-dicaffeoylquinic acid (Sigma-Aldrich). Surprisingly, an increase of phenolic compounds was observed in the chicory KO lines (see FIG. 8). The phenolic and terpene biosynthetic pathways are not directly related and do not source from the same pool of precursor and intermediates therefore the increase of phenolic compounds upon deletion of the GAS gene is unexpected. Chlorogenic acid accumulation was increased 3.8-fold, 3.0-fold and 1.7-fold in the roots of chicory KO lines MT1, MT3 and MT4, respectively. 3,5-dicaffeoylquinic acid was increased 5.6-fold, 4.0-fold and 1.9-fold in the roots of lines MT1, MT3 and MT4, respectively. Wild-type levels of chlorogenic acid and 3,5-dicaffeoylquinic acid were observed in roots of MT2 and MT5 lines. In the leaves increase of phenolic compounds was less pronounced. Increased level of chlorogenic acid was observed in lines MT1, MT2, MT3 up to maximally 2.6-fold in MT3. The content of chicoric acid was similarly increased in the leaves of the chicory KO lines MT1, MT2, MT3.

1. A method for producing a plant having, compared to a control plant, at least one of:
(a) reduced sesquiterpene lactone (STL) level;
(b) increased squalene level; and
(c) increased level of a phenolic compound,
the method comprising mutating one or more endogenous functional germacrene A synthase (GAS)-short genes in the plant, resulting in a decreased or abolished expression or activity of one or more functional GAS-short proteins.

2. The method according to claim 1, wherein the one or more GAS-short genes encode a protein having at least 70% sequence identity with any one of SEQ ID NO: 1-6.

3. The method according to claim 1, wherein multiple, preferably all, endogenous functional GAS-short genes in the plant are mutated.

4. The method according to claim 3, wherein all endogenous functional GAS-short genes in the plant are mutated.

5. The method according to claim 1, wherein at least one nucleotide is inserted, deleted or substituted in a coding sequence of the one or more GAS-short genes, resulting in decreased or abolished activity of an encoded GAS-short proteins.

6. The method according to claim 1, wherein at least one nucleotide is inserted, deleted or substituted in at least one transcription regulatory sequence of the one or more GAS-short genes, resulting in decreased or abolished expression of an encoded GAS-short proteins.

7. The method according to claim 1, wherein the one or more endogenous functional GAS-short genes are any one of CiGAS-S1, CiGAS-S2 and CiGAS-S3, or a homologue thereof.

8. The method according to claim 1, wherein the expression of the protein is impaired in at least any one of the leaves and the roots of the plant.

9. The method according to claim 1, further comprising regenerating the plant by at least one of:
(a) inulin extraction;
(b) squalene extraction; and
(c) phenolic compound extraction,
from the plant.

10. The method according to claim 9, comprising regenerating the plant by at least one of:
(a) inulin extraction;
(b) squalene extraction; and
(c) phenolic compound extraction,
from the plant root.

11. A nucleic acid comprising a germacrene A synthase (GAS)-short gene having one or more modifications, resulting in impaired expression or activity of a functional GAS-short protein when the nucleic acid is present in a plant as compared to an identical nucleic acid not having the one or more modifications.

12. The nucleic acid according to claim 10, wherein the functional GAS-short protein has at least 70% sequence identity with any one of SEQ ID NO: 1-6.

13. A construct, vector or host cell comprising the nucleic acid of claim 9.

14. A plant obtainable from a method according to claim 1, or progeny thereof.

15. A plant having at least one of:
(a) a reduced sesquiterpene lactone (STL) level;
(b) an increased squalene level; and
(c) an increased level of a phenolic compound,
as compared to a control plant, wherein the plant exhibits reduced expression and/or reduced activity of a functional germacrene A synthase (GAS)-short protein, or progeny thereof.

16. The plant according to claim 15, comprising a mutation in one or more endogenous functional GAS-short genes.

17. The plant according to claim 16, comprising a mutation in all of the endogenous functional GAS-short genes.

18. The plant according to claim 15, wherein the functional GAS-short protein has at least 70% sequence identity with any one of SEQ ID NO: 1-6.

19. The plant according to claim 15, comprising a nucleic acid having a germacrene A synthase (GAS)-short gene having one or more modifications.

20. A method of producing at least one of inulin, squalene and a phenolic compound, the method comprising:
(a) providing a plant having at least one of:
(i) reduced sesquiterpene lactone (STL) level;
(ii) increased squalene level; and
(iii) increased level of a phenolic compound,
as compared to a control plant, wherein the plant exhibits reduced expression and/or reduced activity of a functional germacrene A synthase (GAS)-short protein, or progeny thereof;
(b) extracting at least one of inulin, squalene and a phenolic compound from said plant or plant part; and
(c) optionally, purifying at least one of said inulin, squalene and a phenolic compound.


SEQUENCE LISTING (71 sequences)

SEQ ID NO: 1 - protein, 558 aa, Cichorium intybus
MAAVEANGTLQTNTKTTEPVRPLANFPPSVWGDRFLSFSLDTKELEGYAKAMEEPKEELRKLIVDPTMDSNKKLSLIYSVHRLGLAYLFLQEIEAQLDKLFKEFNLQDYDEFDLYTTSINFQVFRHLGHKLPCDVFNKFKDSSLGTFKESITNDVKGMLGLYESAQLRLRGEPILDEASAFTETQLKSVVNTLEGNLAKQVMQSLRRPFHQGMPMVEARMYFSNYDEECSTHESLLKLAKLHFNYLQLQQKEELRIVSKWWKDMRFQETTPYIRDRVPEIYLWILGLYFEPRYSLARIIATKITLFLVVLDDTYDAYATIEEIRLLTDAINRWDISAMEQIPEYIRPFYKILLDEYAELEKQLAKEGRAKSVIASKEAFQDIARGYLEEAEWTNSGYVASFPEYMKNGLITSAYNVISKSALVGMGEMVSEDALAWYESHPKTLQASELISRLQDDVMTYQFERERGQSATGVDSYIKTYGVTEKEAIDELNKMIENAWKDINEGCLKPREVSMDLLAPILNLARMIDVVYRYDDGFTFPGKTMKEYITLLFVGSSPM

SEQ ID NO: 2 - protein, 558 aa, Cichorium intybus
MAAVEANGTLQANTKTTEPVRPLANFPPSVWGDRFLSFSLDTKELEGYAKAMEEPKEELRKLIVDPTMDSKKKLSLIYSVHRLGLAYLFLQEIEAQLDKLFKEFNLQDYDEFDLYTTSINFQVFRHLGHKLPCDVFNKFKDSSLGTFKESITNDVKGMLGLYESAQLRLRGEPILDEASAFTETQLKSVVNTLEGNLAKQVMQSLRRPFHQGMPMVEARMYFSNYDEECSTHESLLKLAKLHFNYLQLQQKEELRIVSKWWKDMRFQETTPYIRDRVPEIYLWILGLYFEPRYSLARIIATKITLFLVVLDDTYDAYATIEEIRLLTDAINRWDISAMEQIPEYIRPFYKILLDEYAELEKQLAKEGRAKSVIASKEAFQDIARGYLEEAEWTNSGYVASFPEYMKNGLITSAYNVISKSALVGMGEMVSEDALAWYESHPKTLQASELISRLQDDVMTYQFERERGQSATGVDSYIKTYGVTEKEAIDELNKMIENAWKDINEGCLKPREVSMDLLAPILNLARMIDVVYRYDDGFTFPGKTMKEYITLLFVGSSPM

SEQ ID NO: 3 - protein, 558 aa, Cichorium intybus
MAAVEANGTFQANTKTTEPVRPLANFPPSVWGDRFLSFSLDTTELEGYAKAMEEPKEEVRKLIVDPTMDSNKKLSLIYSVHRLGLTYLFLQEIEAQLDKLFKEFNLQDYDEFDLYTTSINFQVFRHLGHKLPCDVFNKFKDSSSGTFKESITNDVKGMLGLYESAQLRLRGEPILDEASAFTETQLKSVVNTLEGNLAKQVMQSLRRPFHQGMPMVEARMYFSNYDEECSTHESLPKLAKLHFNYLQLQQKEELRIVSKWWKDMRFQETTPYIRDRVPEIYLWILGLYFEPRYSLARIIATKITLFLVVLDDTYDAYATIEEIRLLTDAINRWDISAMEQIPEYIRPFYKILLDEYAELEKQLAKEGRAKSVIASKEAFQDIARGYLEEAEWTNSGYVASFPEYMKNGLITSAYNVISKSALVGMGEMVGEDALAWYESHPKTLQASELISRLQDDVMTYQFERERGQSATGVDSYIKTYGVTEKEAIDELNKMIENAWKDINEGCLKPREVSMDLLAPILNLARMIDVVYRYDDGFTFPGKTMKEYITLLFVGSSPM

SEQ ID NO: 4 - protein, 558 aa, Cichorium intybus; identical to SEQ ID NO: 3

SEQ ID NO: 5 - protein, 559 aa, Cichorium intybus
MAAVETNGTIQAKTNTTAEPVRPLANFPPSVWGDRFLSFTLDNSELEGYARAMEEPKEDLRRLITDATMESNKKLSLIYSVHRLGLTYLFLQEIEAQLDKLFKEFNLQDYIDLDLYTLSINFQVFRHLGYKLPCDVFNKIKDSNSGTFKEDISTDVKGMLGLYESAQLRVRGESILDEASAFTEAKLKSVVNTLEGNLAKQVMQSLRRPFHQGMPMVEARLYFSNYDEDCSTHESLLKLAKLHFNYLELQQKEELRIVSKWWKDMRFQETTPYIRDRVPEIYLWILGLYFEPRYSLARIIATKITLFLVVLDDTYDAYATIEEIRLLTDAINRWDMSAIEQIPEYIRPFYKILLDEYADLEKQLAIEGRANSVIASKEAFQDIARGYLEEAEWTNSGYVASFPEYMKNGLITSAYNVISKSALVGMGEIVSENALAWYESHPKTLQASELISRLQDDVMTYQFERERGQSATGVDAYIKTYGVSEKEAIDELKIMIENAWKDINEGCLKPREVSMDLLAPILNLARMIDVVYRYDDGFTFPGKTMKEYITLLFVGSVSM

SEQ ID NO: 6 - protein, 559 aa, Cichorium intybus
MAAVETNGTIQAKTNTTAEPVRPLANFPPSVWGDRFLSFTLDNSELEGYARAMEEPKEDLRRLITDATMESNKKLSLIYSVHRLGLTYLFLQEIEAQLDKLFKEFNLQDYIDLDLYTLSINFQVFRHLGYKLPCDVFNKIKVSNSGTFKEDISTDVKGMLGLYESAQLRVRGESILDEASAFTEAKLKSVVNTLEGNLAKQVMQSLRRPFHQGMPMVEARLYFSNYDEDCSTHESLLKLAKLHFNYLELQQKEELRIVSKWWKDMRFQETTPYIRDRVPEIYLWILGLYFEPRYSLARIIATKITLFLVVLDDTYDAYATIEEIRLLTDAINRWDMSAIEQIPEYIRPFYKILLDEYADLEKQLAIEGRANSVIASKEAFQDIARGYLEEAEWTNSGYVASFPEYMKNGLITSAYNVISKSALVGMGEIVSENALAWYESHPKTLQASELISRLQDDVMTYQFERERGQSATGVDAYIKTYGVSEKEAIDELKIMIENAWKDINEGCLKPREVSMDLLAPILNLARMIDVVYRYDDGFTFPGKTMKEYITLLFVGSVSM

SEQ ID NO: 7 - DNA, 1677 bp, Cichorium intybus
atggcagcagttgaagccaacggtaccctccaaacaaacaccaaaaccacagagccagtgcgtcctttagccaacttccctccttctgtatggggtgatcgattcttgtcattctctcttgacactaaggaattggaaggatatgcaaaagctatggaggagccaaaagaagaactgagaaaactgattgtagatccaacaatggattcaaataaaaaactaagtttgatttattctgtacaccgtcttggtttggcatatctgttcttgcaagagattgaagcgcagcttgacaaactatttaaagagtttaacttgcaagattatgatgagtttgatctatacacaacttctattaactttcaagttttcagacaccttggtcacaaactgccatgtgatgtgtttaacaaattcaaggacagtagcttgggtacattcaaggaatctattaccaacgatgtgaagggtatgttaggcttatatgaatctgcacaattgagattaagaggagaacccattctcgatgaagcttccgcattcactgaaactcaactcaagagtgtagtaaacactctcgaaggcaatcttgcaaaacaggtgatgcaatcattgaggagaccattccatcaggggatgccaatggttgaggcaaggatgtatttctccaactatgatgaagaatgttccacacatgagtcattacttaagcttgcaaagcttcatttcaactatttgcagctacaacaaaaggaagaacttcgcattgtctcaaagtggtggaaggatatgaggttccaggaaactactccttacataagggatagagtaccagagatttacttatggatattgggattgtactttgagcctcgttactccttggcacgaatcatcgccacaaaaataacattgtttctggtggtgctagatgacacatatgatgcgtacgctaccattgaagagattcgacttctaacagatgccataaacaggtgggacattagtgctatggagcagattccagaatacattcgaccattctacaaaattctcctagatgagtatgctgaacttgagaagcaactagctaaagaaggaagagcaaaaagtgttattgcttcaaaagaagcgttccaggacattgcaagaggctaccttgaagaggccgagtggacaaacagtggatatgtggcatcatttccagagtatatgaagaacggtttaattacttctgcttacaatgttatttcaaaatctgctttagtgggtatgggcgagatggttagtgaagatgccttggcttggtatgaaagtcatccaaagacactgcaagcttcagagttaatttcaagactccaagacgatgtgatgacttaccagtttgagcgagaaaggggacaatcagccaccggcgttgattcttatatcaagacctacggcgtaacagaaaaggaagcgattgacgagctaaacaaaatgattgaaaatgcatggaaggatataaatgagggctgccttaagccaagagaagtctcaatggatttgcttgccccaattcttaatcttgcacgaatgatagacgtggtatacagatacgacgatgggttcacttttccaggaaagaccatgaaagagtatattactctgttgtttgttggttcttcacccatgtaa

SEQ ID NO: 8 - DNA, 1677 bp, Cichorium intybus
atggcagcagttgaagccaacggtaccctccaagcaaacaccaaaaccacagagccagtgcgtcctttagccaacttccctccttctgtatggggtgatcgattcttgtcattctctcttgacactaaggaattggaaggatatgcaaaagctatggaggagccaaaagaagaactgagaaaactgattgtagatccaacaatggattcaaaaaaaaaactaagtttgatttattctgtacaccgtcttggtttggcatatctgttcttgcaagagattgaagcgcagcttgacaaactatttaaagagtttaacttgcaagattatgatgagtttgatctatacacaacttctattaactttcaagttttcagacaccttggtcacaaactgccatgtgatgtgtttaacaaattcaaggacagtagcttgggtacattcaaggaatctattaccaacgatgtgaagggtatgttaggcttatatgaatctgcacaattgagattaagaggagaacccattctcgatgaagcttccgcattcactgaaactcaactcaagagtgtagtaaacactctcgaaggcaatcttgcaaaacaggtgatgcaatcattgaggagaccattccatcaggggatgccaatggttgaggcaaggatgtatttctccaactatgatgaagaatgttccacacatgagtcattacttaagcttgcaaagcttcatttcaactatttgcagctacaacaaaaggaagaacttcgcattgtctcaaagtggtggaaggatatgaggttccaggaaactactccttacataagggatagagtaccagagatttacttatggatattgggattgtactttgagcctcgttactccttggcacgaatcatcgccacaaaaataacattgtttctggtggtgctagatgacacatatgatgcgtacgctaccattgaagagattcgacttctaacagatgccataaacaggtgggacattagtgctatggagcagattccagaatacattcgaccattctacaaaattctcctagatgagtatgctgaacttgagaagcaactagctaaagaaggaagagcaaaaagtgttattgcttcaaaagaagcgttccaggacattgcaagaggctaccttgaagaggccgagtggacaaacagtggatatgtggcatcatttccagagtatatgaagaacggtttaattacttctgcttacaatgttatttcaaaatctgctttagtgggtatgggcgagatggttagtgaagatgccttggcttggtatgaaagtcatccaaagacactgcaagcttcagagttaatttcaagactccaagacgatgtgatgacttaccagtttgagcgagaaaggggacaatcagccaccggcgttgattcttatatcaagacctacggcgtaacagaaaaggaagcgattgacgagctaaacaaaatgattgaaaatgcatggaaggatataaatgagggctgccttaagccaagagaagtctcaatggatttgcttgccccaattcttaatcttgcacgaatgatagacgtggtatacagatacgacgatgggttcacttttccaggaaagaccatgaaagagtatattactctgttgtttgttggttcttcacccatgtaa

SEQ ID NO: 9 - DNA, 1680 bp, Cichorium intybus
atggcagcagttgaagccaacggtaccttccaagcaaacaccaaaaccacagagccggtgcgtcctttagccaacttccctccttctgtatggggtgatcgattcttgtcattctctcttgacactacggaattggaaggatatgcaaaagctatggaggagccaaaagaagaagtgagaaaactgatcgtagatccaacaatggattcaaataagaaactaagtttgatttattctgtacaccgtcttggtttgacatatctgttcttgcaagagattgaagcgcagcttgacaaactatttaaagagtttaacttgcaagattatgatgagtttgatctatacacaacttctattaactttcaagttttcagacaccttggtcacaaactgccatgtgatgtgtttaacaaattcaaggacagtagctcgggtacattcaaggaatccattaccaacgatgtgaagggtatgttaggcttatatgaatctgcacaattgagattaagaggagaacccattctcgatgaagcttccgcattcactgaaactcaactcaagagtgtagtaaacactctcgaaggaaatcttgcaaaacaggtgatgcaatcattgaggagaccattccatcaggggatgccaatggttgaggcaaggatgtatttctccaactatgatgaagaatgttccacacacgagtcattacctaagcttgcaaagcttcatttcaactatttgcagctacaacaaaaggaagaacttcgcattgtctcaaagtggtggaaggatatgaggttccaggaaactactccttacataagggatagagtaccagagatttacttatggatattgggattgtactttgagcctcgttactccttggcacgaatcatcgccacaaaaattacattgttcctggtggttctagatgacacatatgatgcatacgctaccattgaagagattcgacttctgacagatgccataaacaggtgggacatcagtgctatggagcaaattccggaatatattcgaccattctacaaaattctcctagatgagtatgctgaacttgagaagcaactagctaaagaaggaagagcaaaaagtgtcattgcttcaaaagaagcgttccaggacattgcaagaggataccttgaagaggccgagtggacaaacagtggatatgtggcatcatttccagagtatatgaagaacggtttaattacttctgcttacaatgttatttcaaaatctgctttagtgggtatgggcgagatggttggtgaagatgccttggcttggtatgaaagtcatccaaagacattgcaagcttcagagttaatttcaagactccaagatgatgtgatgacttaccagtttgagcgagaaaggggacaatcagccaccggcgttgattcttatatcaagacctatggcgtaacagaaaaggaagcgattgacgagctcaataaaatgattgaaaatgcatggaaggatataaatgagggctgccttaagccaagagaagtctcaatggatttgcttgccccaattcttaatcttgcacgaatgatagatgtggtatacaggtacgacgatgggttcacttttccgggaaagaccatgaaagagtatattactctgttgtttgttggttcttcacccatgtaataa

SEQ ID NO: 10 - DNA, 1680 bp, Cichorium intybus
atggcagcagttgaagccaacggtaccttccaagcaaacaccaaaaccacagagccggtgcgtcctttagccaacttccctccttctgtatggggtgatcgattcttgtcattctctcttgacactacggaattggaaggatatgcaaaagctatggaggagccaaaagaagaagtgagaaaactgatcgtagatccaacaatggattcaaataagaaactaagtttgatttattctgtacaccgtcttggtttgacatatctgttcttgcaagagattgaagcgcagcttgacaaactatttaaagagtttaacttgcaagattatgatgagtttgatctatacacaacttctattaactttcaagttttcagacaccttggtcacaaactgccatgtgatgtgtttaacaaattcaaggacagtagctcgggtacattcaaggaatccattaccaacgatgtgaagggtatgttaggcttatatgaatctgcacaattgagattaagaggagaacccattctcgatgaagcttccgcattcactgaaactcaactcaagagtgtagtaaacactctcgaaggcaatcttgcaaaacaggtgatgcaatcattgaggagaccattccatcaggggatgccaatggttgaggcaaggatgtatttctccaactatgatgaagaatgttccacacacgagtcattacctaagcttgcaaagcttcatttcaactatttgcagctacaacaaaaggaagaacttcgcattgtctcaaagtggtggaaggatatgaggttccaggaaactactccttacataagggatagagtaccagagatttacttatggatattgggattgtactttgagcctcgttactccttggcacgaatcatcgccacaaaaattacattgttcctcgtggttctagatgacacatatgatgcatacgctaccattgaagagattcgacttctgacagatgccataaacaggtgggacatcagtgctatggagcaaattccggaatatattcgaccattctacaaaattctcctagatgagtatgctgaacttgagaagcaactagctaaagaaggaagagcaaaaagtgttattgcttcaaaagaagcgttccaggacattgcaagaggataccttgaagaggccgagtggacaaacagtggatatgtggcatcatttccagagtatatgaagaacggtttaattacttctgcttacaatgttatttcaaaatctgctttagtgggtatgggcgagatggttggtgaagatgccttggcttggtatgaaagtcatccaaagacattgcaagcttcagagttaatttcaagactccaagatgatgtcatgacttaccagtttgagcgagaaaggggacaatcagccaccggcgttgattcttatatcaagacctatggcgtaacagaaaaggaagcgattgacgagctcaataaaatgattgaaaatgcatggaaggatataaatgagggctgccttaagccaagagaagtctcaatggatttgcttgccccaattcttaatcttgcacgaatgatagatgtggtatacaggtacgacgatgggttcacttttccgggaaagaccatgaaagagtatattactctgttgtttgttggttcttcacccatgtaataa

SEQ ID NO: 11 - DNA, 1680 bp, Cichorium intybus
atggccgcagtcgaaaccaatggtaccatccaagcgaagaccaataccaccgctgagccagtgcgtcccctagccaacttccctccttctgtatggggtgatcgcttcctatcgttcacccttgacaattcggagttggaaggatatgctagagcaatggaagaaccaaaagaagatttgagaagattgatcacagatgcaacaatggaatccaataagaaactaagtttgatatattctgtgcaccgtcttggtttgacatatctattcttgcaagagattgaagcccagctagacaaacttttcaaagagtttaatttgcaagattacattgaccttgatctttatacactttccattaactttcaagttttccgacaccttggttataaactgccttgtgacgtgtttaacaaaatcaaggacagtaactcgggtacattcaaggaagacatttccaccgatgtgaagggaatgttaggcttatatgaatctgcacaactgagagtaagaggagaatcgattctagatgaagcctcagcattcactgaagctaaactcaagagtgtagtaaacactctcgaaggtaatcttgcaaaacaggtgatgcaatctttgagaagaccgttccatcaagggatgcccatggtcgaggcaaggctatatttctccaactatgatgaagattgttctacacacgagtcattacttaagcttgcaaagctccatttcaactatttagagctacaacaaaaggaagaacttcgcattgtctcaaagtggtggaaggatatgaggttccaagaaactactccttatataagggatagagtacccgagatctacttatggatattaggattgtactttgagcctcgttactccttggcacgaatcatcgccacaaaaattacattgtttttggtggtgctagacgacacatatgatgcatatgctacaattgaagaaattcgtcttttaactgatgccataaataggtgggacatgagtgctattgagcaaatcccggaatacattagaccattttacaaaattctcctggatgagtatgctgatcttgagaaacaactagccatagaaggaagagcaaatagcgttatagcttcaaaagaagcgttccaagacattgctagaggttaccttgaagaggctgagtggacaaacagtggatatgtggcatcatttccagagtacatgaagaacggtttaattacttctgcttacaatgttatttcaaaatctgctttagtgggtatgggcgagatagttagtgaaaatgcattagcttggtacgaaagtcatccaaagactctacaagcttccgagttaatctcaagactccaagatgatgtcatgacttaccagtttgagcgtgaaagaggacaatcagccactggcgttgatgcatatatcaagacatatggcgtgtcagagaaggaagctattgacgagctcaagataatgattgaaaatgcatggaaagatataaacgagggatgtctcaagccaagagaagtctccatggatttgttggcgccaattcttaaccttgcaagaatgatagatgttgtgtatagatatgatgacgggttcacttttcccgggaaaaccatgaaagagtatattactcttttatttgttggttctgtatccatgtaa

SEQ ID NO: 12 - DNA, 1680 bp, Cichorium intybus
atggccgcagtcgaaaccaatggtaccatccaagcgaagaccaataccaccgctgagccagtgcgtcccctagccaacttccctccttctgtatggggtgatcgcttcctatcgttcacccttgacaattcggagttggaaggatatgctagagcaatggaagaaccaaaagaagatttgagaagattgatcacagatgcaacaatggaatccaataagaaactaagtttgatatattctgtgcaccgtcttggtttgacatatctattcttgcaagagattgaagcccagctagacaaacttttcaaagagttcaatttgcaagattacattgaccttgatctttatacactttccattaactttcaagttttccgacaccttggttataaactgccttgtgacgtgtttaacaaaatcaaggtcagtaactcgggtacattcaaggaagacatttccaccgatgtgaagggaatgttaggcttatatgaatctgcacaactgagagtaagaggagaatcgattctagatgaagcctcagcattcactgaagctaaactcaagagtgtagtaaacactctcgaaggtaatcttgcaaaacaggtgatgcaatctttgagaagaccgttccatcaagggatgcccatggtcgaggcaaggctatatttctccaactatgatgaagattgttctacacacgagtcattacttaagcttgcaaagctccatttcaactatttagagctacaacaaaaggaagaacttcgcattgtctcaaagtggtggaaggatatgaggttccaagaaactactccttatataagggatagagtacccgagatctacttatggatattaggattgtactttgagcctcgttactccttggcacgaatcatcgccacaaaaattacattgtttttggtggtgctagacgacacatatgatgcatatgctacaattgaagaaattcgtcttttaactgatgccataaataggtgggacatgagtgctattgagcaaatcccggaatacattagaccattttacaaaattctcctggatgagtatgctgatcttgagaaacaactagccatagaaggaagagcaaatagcgttatagcttcaaaagaagcgttccaagacattgctagaggttaccttgaagaggctgagtggacaaacagtggatatgtggcatcatttccagagtacatgaagaacggtttaattacttctgcttacaatgttatttcaaaatctgctttagtgggtatgggcgagatagttagtgaaaatgcattagcttggtacgaaagtcatccaaagactctacaagcttccgagttaatctcaagactccaagatgatgtcatgacttaccagtttgagcgtgaaagaggacaatcagccactggcgttgatgcatatatcaagacatatggcgtgtcagagaaggaagctattgacgagctcaagataatgattgaaaatgcatggaaagatataaacgagggatgtctcaagccaagagaagtctccatggatttgttggcgccaattcttaaccttgcaagaatgatagatgttgtgtatagatatgatgacgggttcacttttcccgggaaaaccatgaaagagtatattactcttttatttgttggttctgtatccatgtaa

SEQ ID NO: 13 - protein, 583 aa, Cichorium intybus
MALVRNNSSNGREPVLSPRSLTSPRGLTSPRPLSVQPTPEPVRPLANFPPSIWADRFISFSLDNSQLEAYANALEEPKEAVKSLITDTTIDANTKLKLIYSVHRLGLSYLYPDEIDAELNKLFEKIDLQYYEQVDLYTIAVQFQVFRHHGYKISSDVFKKFKDSTTGTFTDDVTKDVKGMLSLYESAHLRLHGEDILDEALAFTEAHLKKIVTTLEGDLARQVNQVLKRPFHTGMPMVEARLYFITHEEDFSSHESVVKLAKVHFNYLQLQQKEELRLVSQWWKDMQFQQSVPYIRDRVPEIYLWILGLYFEPYYSRARIIATKITLFLVVLDDTYDAYATIDEIRSITDAINRWEISAIDQLPEYIKPFYRILLNEYDDLEKEYSKDGRAFSVHASKQAFQEIARGYLEEAEWLHNGYVATFPEYMKNGLITSAYNVISKSALVGMGAIADEEALAWYETHPKILKASELISRLQDDVMTFQFERKRGQSATGVDAYIKEYNVSEEVAIQELMKMIENAWKDINEGCLKPTEVSVALLTPILNLARMIDVVYKFDDGFTFPGKTLKDYITLLFVSPPPSLEN

SEQ ID NO: 14 - DNA, 1752 bp, Cichorium intybus
atggctctcgttagaaacaacagcagcaatggtcgggagccagttctcagccccagaagtctcacaagtcctcgaggcttaaccagcccccgaccgttgtctgtccaaccgactccagagccggttcgacctttggccaacttcccaccttcgatctgggctgatcgcttcatctcattctctcttgataactctcaattggaagcttatgcaaatgcacttgaagaaccaaaagaagcagtgaagagtttaataaccgacactaccattgatgcaaacacaaaactgaaattgatttattcagtgcaccgtcttggtttgtcgtatctttatccagatgagattgatgccgaactcaacaaactcttcgagaagattgacttacagtattacgaacaagttgatttgtacactattgcagtacaatttcaagttttcagacaccatggttataaaatttcttctgatgtgtttaaaaagttcaaggacagtactacgggtacattcacggatgatgtaacaaaagacgtgaagggtatgttgagtctatacgaatcagcacacttaaggctacacggtgaagatatcttagatgaagctttagcattcaccgaagctcaccttaaaaaaatcgtaaccacacttgaaggagatcttgcacgccaagtgaaccaagtcttaaaaagacccttccacactggaatgccaatggtagaggcacggctatatttcatcacacacgaagaagatttttcaagccatgagtcggttgtaaagctagctaaagtccacttcaactatttgcaactacaacaaaaagaagaactacgactcgtgtcacagtggtggaaagatatgcaattccaacaatcggtcccttacataagagatagagtaccggagatatacctatggattttggggttatatttcgagccgtattactctcgggcacgtatcatagccactaaaatcacgttgttcttggtggttttagatgatacgtatgacgcgtatgctacaattgacgagatccgatcgatcacagatgcgattaataggtgggaaattagcgcgatcgaccaacttcctgaatatatcaaaccgttctacagaattctcctcaacgaatacgatgacctcgagaaagaatactccaaggacggaagagcgttcagtgtccacgcttcaaaacaagcgtttcaagaaatcgcgagagggtatcttgaagaggcggagtggttacacaacggttatgtggcaacatttcccgagtatatgaagaatggtttgattacttcggcttataatgtcatttcaaaatccgcattggtgggaatgggtgcgattgcagatgaagaggctcttgcttggtatgaaacacatccgaaaattttgaaagcttcagagttgatttcaaggctccaagacgatgttatgactttccagtttgagagaaaacgaggacaatcagcaactggtgtggatgcttatatcaaggaatacaatgtatccgaagaagtggcgatccaagagctcatgaagatgattgaaaacgcatggaaagatataaatgagggatgcttgaagcctacagaggtctcggtggctttactaactcctattttgaatctcgcgagaatgatagatgtcgtatacaaatttgatgatgggttcacctttcccgggaaaaccctaaaagactatattacccttttgttcgttagtcctccacctagtctcgaaaattga

SEQ ID NO: 15 - protein, 40 aa, Artificial Sequence
  N-terminal sequence of GAS-short protein
MAAVEANGTFQANTKTTEPVRPLANFPPSVWGDRFLSFSL

SEQ ID NO: 16 - protein, 40 aa, Artificial Sequence
  N-terminal sequence of GAS-long protein
MALVRNNSSNGREPVLSPRSLTSPRGLTSPRPLSVQPTPE

SEQ ID NO: 17 - DNA, 103 bp, Artificial Sequence
  sgRNA targeting GAS-S1 and GAS-S2
ggtactctatcccttatgtagttttagagctagaaatagcaagttaaaataaggctagtccgttatcaacttgaaaaagtggcaccgagtcggtgcttttttt

SEQ ID NO: 18 - DNA, 103 bp, Artificial Sequence
  sgRNA targeting GAS-S3
ggtactctatcccttatatagttttagagctagaaatagcaagttaaaataaggctagtccgttatcaacttgaaaaagtggcaccgagtcggtgcttttttt

SEQ ID NO: 19 - DNA, 103 bp, Artificial Sequence
  sgRNA targeting GAS-L1
ggtactctatctcttatgtagttttagagctagaaatagcaagttaaaataaggctagtccgttatcaacttgaaaaagtggcaccgagtcggtgcttttttt

SEQ ID NO: 20 - DNA, 183 bp, Artificial Sequence
  Expression cassette encoding sgRNA targeting GAS-S1 and GAS-S2
ggagtgatcaaaagtcccacatcgatcaggtgatatatagcagcttagtttatataatgatagagtcgacatagcgattgggtactctatcccttatgtagttttagagctagaaatagcaagttaaaataaggctagtccgttatcaacttgaaaaagtggcaccgagtcggtgcttttttt

SEQ ID NO: 21 - DNA, 608 bp, Artificial Sequence
  Expression cassette comprising three sgRNAs targeting GAS-S1, GAS-S2, GAS-S3 and GAS-L1
ggagtgatcaaaagtcccacatcgatcaggtgatatatagcagcttagtttatataatgatagagtcgacatagcgattgaacaaagcaccagtggtctagtggtagaatagtaccctgccacggtacagacccgggttcgattcccggctggtgcaggtactctatcccttatgtagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggtactctatcccttatatagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggtactctatctcttatgtagttttagagctagaaatagcaagttaaaataaggctagtccgttatcaacttgaaaaagtggcaccgagtcggtgcttttttttt

SEQ ID NO: 22 - DNA, 23 bp, Artificial Sequence
  Target sequence of GAS-S1and GAS-S2
ggtactctatcccttatgtaagg

SEQ ID NO: 23 - DNA, 23 bp, Artificial Sequence
  Target sequence of GAS-S3
ggtactctatcccttatataagg

SEQ ID NO: 24 - DNA, 23 bp, Artificial Sequence
  Target sequence of GAS-L1
ggtactctatctcttatgtaagg

SEQ ID NO: 25 - DNA, 22 bp, Artificial Sequence
  GAS-S1 (-1)
ggtactctatcccttattaagg

SEQ ID NO: 26 - DNA, 19 bp, Artificial Sequence
  GAS-S1 (-4)
ggtactctatcctgtaagg

SEQ ID NO: 27 - DNA, 16 bp, Artificial Sequence
  GAS-S1 (-7)
ggtactctatgtaagg

SEQ ID NO: 28 - DNA, 14 bp, Artificial Sequence
  GAS-S1 (-9)
ggtactctataagg

SEQ ID NO: 29 - DNA, 11 bp, Artificial Sequence
  GAS-S1 (-12)
ggtacgtaagg

SEQ ID NO: 30 - DNA, 9 bp, Artificial Sequence
  GAS-S1 (-14)
ggtgtaagg

SEQ ID NO: 31 - DNA, 5 bp, Artificial Sequence
  GAS-S1 (-19)
taagg

SEQ ID NO: 32 - DNA, 24 bp, Artificial Sequence
  GAS-S2 (+1)
ggtactctatcccttattgtaagg

SEQ ID NO: 33 - DNA, 19 bp, Artificial Sequence
  GAS-S2 (-4)
ggtactctatcccgtaagg

SEQ ID NO: 34 - DNA, 16 bp, Artificial Sequence
  GAS-S2 (-7)
ggtactctatgtaagg

SEQ ID NO: 35 - DNA, 13 bp, Artificial Sequence
  GAS-S2 (-10)
ggtactctatagg

SEQ ID NO: 36 - DNA, 10 bp, Artificial Sequence
  GAS-S2 (-13)
ggttgtaagg

SEQ ID NO: 37 - DNA, 21 bp, Artificial Sequence
  GAS-S3 (-2)
ggtactctatcccttataagg

SEQ ID NO: 38 - DNA, 19 bp, Artificial Sequence
  GAS-S2 (-4)
ggtactctatcccataagg

SEQ ID NO: 39 - DNA, 16 bp, Artificial Sequence
  GAS-S3 (-7)
ggtactctatataagg

SEQ ID NO: 40 - DNA, 14 bp, Artificial Sequence
  GAS-S3 (-9)
ggtactctataagg

SEQ ID NO: 41 - DNA, 7 bp, Artificial Sequence
  GAS-S3 (-16)
ggtaagg

SEQ ID NO: 42 - DNA, 22 bp, Artificial Sequence
  GAS-L1 (-1)
ggtactctatctcttagtaagg

SEQ ID NO: 43 - DNA, 18 bp, Artificial Sequence
  GAS-L1 (-5)
ggtactctatctgtaagg

SEQ ID NO: 44 - DNA, 17 bp, Artificial Sequence
  GAS-L1 (-6)
ggtactctattgtaagg

SEQ ID NO: 45 - DNA, 20 bp, Artificial Sequence
  GAS-S1 F primer
tgtaaaccgttgggctcgtc

SEQ ID NO: 46 - DNA, 22 bp, Artificial Sequence
  GAS-S2 F primer
ctattaatcggctttggtcaac

SEQ ID NO: 47 - DNA, 22 bp, Artificial Sequence
  GAS-S3 F primer
gttatttcagttccccaagtgt

SEQ ID NO: 48 - DNA, 18 bp, Artificial Sequence
  GAS-L F primer
cgtacgtagagtgtccaa

SEQ ID NO: 49 - DNA, 19 bp, Artificial Sequence
  GAS-S1 R primer
gagtttatagcacatgcgt

SEQ ID NO: 50 - DNA, 20 bp, Artificial Sequence
  GAS-S2 R primer
ccatatggactcgctcaaac

SEQ ID NO: 51 - DNA, 21 bp, Artificial Sequence
  GAS-S3 R primer
gtgtttttgtcctatccgtct

SEQ ID NO: 52 - DNA, 19 bp, Artificial Sequence
  GAS-L R primer
cccaaaagaagcccaatcc

SEQ ID NO: 53 - DNA, 51 bp, Artificial Sequence
  GAS-S1/S2 nF primer
actctttccctacacgacgctcttccgatcttatgaggttccaggaaacta

SEQ ID NO: 54 - DNA, 51 bp, Artificial Sequence
  GAS-S3 nF primer
actctttccctacacgacgctcttccgatcttatgaggttccaagaaacta

SEQ ID NO: 55 - DNA, 51 bp, Artificial Sequence
  GAS-L nF primer
actctttccctacacgacgctcttccgatcttatgcaattccaacaatccg

SEQ ID NO: 56 - DNA, 48 bp, Artificial Sequence
  GAS-S1/S2 nR primer
tggagttcagacgtgtgctcttccgatctaggctcaaagtacaatccc

SEQ ID NO: 57 - DNA, 48 bp, Artificial Sequence
  GAS-S3 nR primer
tggagttcagacgtgtgctcttccgatctaggctcaaagtacaatcct

SEQ ID NO: 58 - DNA, 49 bp, Artificial Sequence
  GAS-L nR primer
tggagttcagacgtgtgctcttccgatctacggctcgaaatataacccc

SEQ ID NO: 59 - DNA, 77 bp, Artificial Sequence
  tRNA
aacaaagcaccagtggtctagtggtagaatagtaccctgccacggtacagacccgggttcgattcccggctggtgca

SEQ ID NO: 60 - DNA, 80 bp, Artificial Sequence
  arabidopsis U6 promoter
ggagtgatcaaaagtcccacatcgatcaggtgatatatagcagcttagtttatataatgatagagtcgacatagcgattg

SEQ ID NO: 61 - protein, 1368 aa, artificial sequence [sequence omitted]

SEQ ID NO: 62 - DNA, 4104 bp, artificial sequence
  sequence encoding Cas9
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 63 - DNA, 80 bp, Artificial Sequence
  sgRNA scaffold
gttttagagctagaaatagcaagttaaaataaggctagtccgttatcaacttgaaaaagtggcaccgagtcggtgctttt

SEQ ID NO: 64 - DNA, 22 bp, Artificial Sequence
  crRNA scaffold
gttttagagctgtgttgtttcg

SEQ ID NO: 65 - DNA, 73 bp, Artificial Sequence
  tracrRNA
cgaaacagcatagcaagttaaaataaggctagtccgttatcaacttgaaaaagtggcaccgagtcggtgcttt

SEQ ID NO: 66 - DNA, 972 bp, Artificial Sequence
  arabidopsis ubiquitin promoter
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagc

SEQ ID NO: 67 - DNA, 220 bp, Cichorium intybus
gtggtggaaggatatgaggttccaggaaactactccttacataagggatagagtaccagagatttacttatggatattgggattgtactttgagcctcgttactccttggcacgaatcatcgccacaaaaataacattgtttctggtggtgctagatgacacatatgatgcgtacgctaccattgaagagattcgacttctaacagatgccataaacagg

SEQ ID NO: 68 - DNA, 220 bp, Cichorium intybus
gtggtggaaggatatgaggttccaggaaactactccttacataagggatagagtaccagagatttacttatggatattgggattgtactttgagcctcgttactccttggcacgaatcatcgccacaaaaattacattgttcctggtggttctagatgacacatatgatgcatacgctaccattgaagagattcgacttctgacagatgccataaacagg

SEQ ID NO: 69 - DNA, 220 bp, Cichorium intybus
gtggtggaaggatatgaggttccaagaaactactccttatataagggatagagtacccgagatctacttatggatattaggattgtactttgagcctcgttactccttggcacgaatcatcgccacaaaaattacattgtttttggtggtgctagacgacacatatgatgcatatgctacaattgaagaaattcgtcttttaactgatgccataaatagg

SEQ ID NO: 70 - DNA, 220 bp, Cichorium intybus
gtggtggaaagatatgcaattccaacaatcggtcccttacataagagatagagtaccggagatatacctatggattttggggttatatttcgagccgtattactctcgggcacgtatcatagccactaaaatcacgttgttcttggtggttttggacgatacatatgacgcgtatgctacaattgacgagatccgatcgatcacagatgcgattaatagg

SEQ ID NO: 71 - DNA, 12 bp, Artificial sequence
  GAS-S3 (-11)
ggtactataagg